SEQUENCE LISTING (21 sequences)

SEQ ID NO: 1 - DNA, 5300 bp, Homo sapiens
atctttgttcagtttacctcagggctattatgaaatgaaatgagataaccaatgtgaaagtcctataaactgtatagcctccattcggatgtatgtctttggcaggatgataaagaatcaggaagaaggagtatccacgttagccaagtgtccaggctgtgtctgctcttattttagtgacagatgttgctcctgacagaagctattcttcaggaaacatcacatccaatatggtaaatccatcaaacaggagctaagaaacaggaatgagatgggcacttgcccaaggaaaaatgccaggagagcaaataatgatgaaaaataaacttttccctttgtttttaatttcaggaaaaaatgatgaggaccaaaatcaatgaataaggaaaacagctcagaaaaaagatgtttccaaattggtaattaagtatttgttccttgggaagagacctccatgtgagcttgatgggaaaatgggaaaaacgtcaaaagcatgatctgatcagatcccaaagtggattattattttaaaaaccagatggcatcactctggggaggcaagttcaggaaggtcatgttagcaaaggacataacaataacagcaaaatcaaaattccgcaaatgcaggaggaaaatggggactgggaaagctttcataacagtgattaggcagttgaccatgttcgcaacacctccccgtctataccagggaacacaaaaattgactgggctaagcctggactttcaagggaaatatgaaaaactgagagcaaaacaaaagacatggttaaaaggcaaccagaacattgtgagccttcaaagcagcagtgcccctcagcagggaccctgaggcatttgcctttaggaaggccagttttcttaaggaatcttaagaaactcttgaaagatcatgaattttaaccattttaagtataaaacaaatatgcgatgcataatcagtttagacatgggtcccaattttataaagtcaggcatacaaggataacgtgtcccagctccggataggtcagaaatcattagaaatcactgtgtccccatcctaactttttcagaatgatctgtcatagccctcacacacaggcccgatgtgtctgacctacaaccacatctacaacccaagtgcctcaaccattgttaacgtgtcatctcagtaggtcccattacaaatgccacctcccctgtgcagcccatcccgctccacaggaagtctccccactctagacttctgcatcacgatgttacagccagaagctccgtgagggtgagggtctgtgtcttacacctacctgtatgctctacacctgagctcactgcaacctctgcctcccaggttcaagcaattctcctgtctcagcctcccgcgtagctgggactacaggcgcacgcccggctaatttttgtattgttagtagagatggggtttcaccatattagcccggctggtcttgaactcctgacctcaggtgatccacccacctcagcctcctaaagtgctgggattacaggcatgagtcaccgcgcccggccaagggtcagtgtttaataaggaataacttgaatggtttactaaaccaacagggaaacagacaaaagctgtgataatttcagggattcttgggatggggaatggtgccatgagctgcctgcctagtcccagaccactggtcctcatcactttcttccctcatcctcattttcaggctaagttaccattttattcaccatgcttttgtggtaagcctccacatcgttactgaaataagagtatacataaactagttccatttggggccatctgtgtgtgtgtataggggaggagggcataccccagagactccttgaagcccccggcagaggtttcctctccagctgggggagccctgcaagcacccggggtcctgggtgtcctgagcaacctgccagcccgtgccactggttgttttgttatcactctctagggacctgttgctttctatttctgtgtgactcgttcattcatccaggcattcattgacaatttattgagtacttatatctgccagacaccagagacaaaatggtgagcaaagcagtcactgccctaccttcgtggaggtgacagtttctcatggaagacgtgcagaagaaaattaatagccagccaacttaaacccagtgctgaaagaaaggaaataaacaccatcttgaagaattgtgcgcagcatcccttaacaaggccacctccctagcgccccctgctgcctccatcgtgcccggaggcccccaagcccgagtcttccaagcctcctcctccatcagtcacagcgctgcagctggcctgcctcgcttcccgtgaatcgtcctggtgcatctgagctggagactccttggctccaggctccagaaaggaaatggagagggaaactagtctaacggagaatctggaggggacagtgtttcctcagagggaaaggggcctccacgtccaggagaattccaggaggtggggactgcagggagtggggacgctggggctgagcgggtgctgaaaggcaggaaggtgaaaagggcaaggctgaagctgcccagatgttcagtgttgttcacggggctgggagttttccgttgcttcctgtgagcctttttatcttttctctgcttggaggagaagaagtctatttcatgaagggatgcagtttcataaagtcagctgttaaaattccagggtgtgcatgggttttccttcacgaaggcctttatttaatgggaatataggaagcgagctcatttcctaggccgttaattcacggaagaagtgactggagtcttttctttcatgtcttctgggcaactactcagccctgtggtggacttggcttatgcaagacggtcgaaaaccttggaatcaggagactcggttttctttctggttctgccattggttggctgtgcgaccgtgggcaagtgtctctccttccctgggccatagtcttctctgctataaagacccttgcagctctcgtgttctgtgaacacttccctgtgattctctgtgaggggggatgttgagaggggaaggaggcagagctggagcagctgagccacaggggaggtggagggggacaggaaggcaggcagaagctgggtgctccatcagtcctcactgatcacgtcagactccaggaccgagagccacaatgcttcaggaaagctcaatgaacccaacagccacattttccttccctaagcatagacaatggcatttgccaataaccaaaaagaatgcagagactaactggtggtagcttttgcctggcattcaaaaactgggccagagcaagtggaaaatgccagagattgttaaacttttcaccctgaccagcaccccacgcagctcagcagtgactgctgacagcacggagtgacctgcagcgcaggggaggagaagaaaaagagagggatagtgtatgagcaagaaagacagattcattcaagggcagtgggaattgaccacagggattatagtccacgtgatcctgggttctaggaggcagggctatattgtggggggaaaaaatcagttcaagggaagtcgggagacctgatttctaatactatatttttcctttacaagctgagtaattctgagcaagtcacaaggtagtaactgaggctgtaagattacttagtttctccttattaggaactctttttctctgtggagttagcagcacaagggcaatcccgtttcttttaacaggaagaaaacattcctaagagtaaagccaaacagattcaagcctaggtcttgctgactatatgattggttttttgaaaaatcatttcagcgatgtttactatctgattcagaaaatgagactagtaccctttggtcagctgtaaacaaacacccatttgtaaatgtctcaagttcaggcttaactgcagaaccaatcaaataagaatagaatctttagagcaaactgtgtttctccactctggaggtgagtctgccagggcagtttggaaatatttacttcacaagtattgacactgttgttggtattaacaacataaagttgctcaaaggcaatcattatttcaagtggcttaaagttacttctgacagttttggtatatttattggctattgccatttgctttttgttttttctctttgggtttattaatgtaaagcagggattattaacctacagtccagaaagcctgtgaatttgaatgaggaaaaaattacatttttgtttttaccaccttctaactaaatttaacattttattccattgcgaatagagccataaactcaaagtggtaataacagtacctgtgattttgtcattaccaatagaaatcacagacattttatactatattacagttgttgcagatacgttgtaagtgaaatatttatactcaaaactactttgaaattagacctcctgctggatcttgtttttaacatattaataaaacatgtttaaaattttgatattttgataatcatatttcattatcatttgtttcctttgtaatctatattttatatatttgaaaacatctttctgagaagagttccccagatttcaccaatgaggttcttggcatgcacacacacagagtaagaactgatttagaggctaacattgacattggtgcctgagatgcaagactgaaattagaaagttctcccaaagatacacagttgttttaaagctaggggtgaggggggaaatctgccgcttctataggaatgctctccctggagcctggtagggtgctgtccttgtgttctggctggctgttatttttctctgtccctgctacgtcttaaaggacttgtttggatctccagttcctagcatagtgcctggcacagtgcaggttctcaatgagtttgcagagtgaatggaaatataaactagaaatatatccttgttgaaatcagcacaccagtagtcctggtgtaagtgtgtgtacgtgtgtgtgtgtgtgtgtgtgtgtgtgtaaaaccaggtggagatataggaactattattggggtatgggtgcataaattgggatgttctttttaaaaagaaactccaaacagacttctggaaggttattttctaagaatcttgctggcagcgtgaaggcaacccccctgtgcacagccccacccagcctcacgtggccacctctgtcttcccccatgaagggctggctccccagtatatataaacctctctggagctcgggcatgagccagcaaggccacccatccaggcacctctcagcacagc

SEQ ID NO: 2 - DNA, 19 bp, synthetic
tgaggcttcctctggaaac

SEQ ID NO: 3 - DNA, 20 bp, synthetic
tgaaatcagcacaccagtag

SEQ ID NO: 4 - DNA, 21 bp, synthetic
gcacccataccccaataatag

SEQ ID NO: 5 - DNA, 20 bp, synthetic
agagttccccagatttcacc

SEQ ID NO: 6 - DNA, 20 bp, synthetic
atctggggaactcttctcag

SEQ ID NO: 7 - DNA, 19 bp, synthetic
tacagttgttgcagatacg

SEQ ID NO: 8 - DNA, 21 bp, synthetic
acaacgtatctgcaacaactg

SEQ ID NO: 9 - DNA, 20 bp, synthetic
tcaggcttaactgcagaacc

SEQ ID NO: 10 - DNA, 19 bp, synthetic
ttggttctgcagttaagcc

SEQ ID NO: 11 - DNA, 19 bp, synthetic
agcagcacaagggcaatcc

SEQ ID NO: 12 - DNA, 18 bp, synthetic
acagggctatattgtggg

SEQ ID NO: 13 - DNA, 19 bp, synthetic
cctgagatgccagctgtcc

SEQ ID NO: 14 - DNA, 20 bp, synthetic
ctgaagcattagaagccaac

SEQ ID NO: 15 - DNA, 20 bp, synthetic
accttggaccaggctgccag

SEQ ID NO: 16 - DNA, 19 bp, synthetic
aggtttgttcgagttccag

SEQ ID NO: 17 - DNA, 20 bp, synthetic
acaattactggcaagtatgg

SEQ ID NO: 18 - DNA, 19 bp, synthetic
ccttctcagccttgctacc

SEQ ID NO: 19 - DNA, 20 bp, synthetic
acacctcagcagatgctacc

SEQ ID NO: 20 - DNA, 19 bp, synthetic
atggatgactgacatggcc

SEQ ID NO: 21 - DNA, 19 bp, synthetic
aaggatgaacatggtcacc

(19) United States
(12) Patent Application Publication
(10) Pub. No.: US 2004/0132795 A1
(43) Pub. Date: Jul. 8, 2004

(54) Methods to screen and treat individuals with glaucoma or the propensity to develop glaucoma

(76) Inventor: Jon Polansky, Mill Valley, CA (US)

Correspondence Address: ARNOLD & PORTER LLP, ATTN: IP DOCKETING DEPT., 555 TWELFTH STREET, N.W., WASHINGTON, DC, 20004-1206, US

(21) Appl. No.: 10/741,339

(22) Filed: Dec. 22, 2003

Related U.S. Application Data
(63) Continuation of application No. 09/985,637, filed on Nov. 5, 2001, now abandoned.

(51) Int. Cl. A61K 31/415; A61K 31/60; C12Q 1/68

(52) U.S. Cl. 514/406; 514/165; 514/420; 514/570; 435/6

(57) ABSTRACT

The present invention involves methods and reagents for diagnosing and treating glaucoma and related disorders. Specifically, the invention relates to a method of identifying mutations in the TIGR gene of a glaucomatous patient and treating them with an effective amount of a non-steroidal anti-inflammatory drug. Additionally the invention allows the identification of individuals at risk for progressive increases in intraocular pressure, which is a risk factor for glaucoma; the invention thus also allows the identification of individuals among ocular hypertensive/glaucoma suspect groups at increased risk of visual field loss.

This application is a continuation of U.S. patent application Ser. No. 09/985,637, filed Nov. 5, 2001.

### FIELD OF THE INVENTION

The present invention involves methods and reagents for diagnosing and treating glaucoma and related disorders. Specifically, the invention relates to a method of identifying mutations in the TIGR gene of a glaucomatous patient and treating them with an effective amount of a non-steroidal anti-inflammatory drug. Additionally the invention allows the identification of individuals at risk for progressive increases in intraocular pressure, which is a risk factor for glaucoma; the invention thus also allows the identification of individuals among ocular hypertensive/glaucoma suspect groups at increased risk of visual field loss.

### BACKGROUND OF THE INVENTION

“Glaucomas” are a group of debilitating eye diseases, which are the leading cause of preventable blindness in the United States and other developed nations. Glaucoma is the second leading cause of permanent blindness in the United States and the single leading cause of blindness among African-Americans (Leske, M. C., American Journal of Epidemiology 118:166-191 (1983), the entirety of which is herein incorporated by reference; Francois, J., Am J. Ophthalmol 61:652-665 (1966), the entirety of which is herein incorporated by reference; Hoskins, H. D. et al., Sixth ed. St. Louis: C. V. Mosby (1989), the entirety of which is herein incorporated by reference).

Historically, one way in which glaucomas have been characterized is by age of onset. Those developing between birth and age three were termed primary infantile glaucoma. The majority of cases of glaucoma develop in adulthood after age forty and have been termed chronic adult glaucoma, among other names that relate to suspected etiologies. Juvenile glaucoma occurs later than infantile glaucoma but earlier than the usual adult forms (Hoskins, H. D. et al., Sixth ed. St. Louis: C. V. Mosby (1989)).

Infantile glaucoma is thought to be caused by incomplete development of the anterior segment of the eye. In contrast, there are no developmental anomalies associated with the more prevalent adult forms of glaucoma. Children with infantile glaucoma typically have symptoms of tearing, photophobia, corneal clouding, and large eyes by the time they reach one year of age.

Juvenile open angle glaucoma usually occurs after age three but before age forty. The two forms of juvenile glaucoma recognized previously are a late form of infantile glaucoma with similar iridocomeal angle anomalies, and another that has normal angles that appears similar to adult primary open angle glaucoma.

Adult onset glaucomas are most often characterized by reduction of the outflow of aqueous humor through the trabecular meshwork, which is located in the angle between the iris and cornea (see, Vaughan, D. et al., In: General Ophthalmology, Appleton & Lange, Norwalk, Conn., pp. 213-230 (1992), the entirety of which is herein incorporated by reference). The obstruction involving this major pathway by which aqueous humor normally leaves the eye can lead to elevated intraocular pressure (IOP), and also represents a major risk factor for developing visual field loss in glaucoma.

In a normal eye, aqueous humor, a clear, nutrient-rich fluid, passes continuously through the pupil and into a small space at the front of the eye, called the anterior chamber. As it leaves this area, the aqueous humor flows to the periphery of the chamber, or angle, where it exits through a complex channel system and drains into blood vessels in and near the sclera, the white outer coat of the eye. In an eye with open-angle glaucoma, the aqueous humor drains too slowly through the major tissue in the outflow channel system (i.e., the trabecular meshwork or TM), creating a chronic rise in fluid pressure inside the eye. This elevated pressure may gradually interrupt the metabolic processes of cells in the optic nerve, leading to a progressive destruction of nerve fibers that are essential for vision. Therefore, a symptom of such obstruction in this disease is an increased IOP, that is a major risk factor in producing progressive visual loss and blindness if not treated appropriately.

At its onset, open-angle glaucoma usually has no symptoms. There is no pain, no blurring of vision, and no ocular inflammation to alert someone that he or she has the disease. But, as open-angle glaucoma progresses, it will slowly begin to destroy peripheral vision. It is advantageous to have a diagnosis of the glaucoma as early as possible and certainly before major visual loss has occurred. Vision that has already been lost from glaucoma is usually irreversible.

The adult onset glaucomas are subdivided by the mechanisms of pressure elevation into closed angle and open angle glaucoma. If the trabecular meshwork is free from mechanical obstruction, the glaucoma is termed primary open angle glaucoma (POAG). Adult primary open angle glaucoma accounts for about 60-70% of all cases of glaucoma (Hoskins, H. D. et al., Sixth ed. St. Louis: C. V. Mosby (1989)). Narrow-angle glaucoma occurs far less frequently than POAG, seen in less than 10 percent of glaucoma patients. In this form of the disease, aqueous humor cannot drain out of the eye due to very narrow drainage angles, which are usually blocked by the iris. This condition can occur slowly and progressively, or very quickly. Closed-angle glaucoma may also be triggered by anything dilating the iris, resulting in more of the iris blocking the angles. Pigmentary glaucoma, another form of the disease associated with pigment from the iris entering and possibly blocking the draining angles, or producing additional changes relating to alteration of the drainage system and reduced outflow. Secondary glaucoma develops after trauma to the eye that affects the drainage system. Injury, infection, inflammation, tumor or an enlarged cataract can precipitate secondary glaucoma.

Glaucoma is estimated to affect between 0.4% and 3.3% of all adults over 40 years old (Leske, M. C. et al., Amer. J. Epidemiol. 113:1843-1846 (1986), the entirety of which is herein incorporated by reference; Bengtsson, B., Br. J. Ophthamol. 73:483-487 (1989), the entirety of which is herein incorporated by reference; Strong, N. P., Ophthal. Physiol. Opt. 12:3-7 (1992), the entirety of which is herein incorporated by reference). Moreover, the prevalence of the disease rises with age to over 6% of those 75 years or older (Strong, N. P., Ophthal. Physiol. Opt. 12:3-7 (1992)).

The TM cells of the outflow pathway are believed to play important roles in the maintenance of the channels for the draining of aqueous humor (Polansky and Alvarado, Cellular mechanisms influencing the aqueous humor outflow pathway, In Albert D M. Jacobiec F A, eds. Priciples and practice of ophthalmology: basic sciences, Philadelphia, W B Saunders, pp 226-51 (1994)).

As described, human trabecular meshwork (HTM) cells are endothelial-like cells which line the outflow channels through which aqueous humor exits the eye. Altered synthetic function of the HTM cells produced in response to pathogenic agents may be involved in the obstruction to outflow seen in steroid glaucoma and other types of glaucoma. Sustained steroid treatment of these cells is interesting because major differences are observed when compared to 1-2 day glucocorticoid (GC) exposure, which appears relevant to the clinical onset of steroid glaucoma (1-6 weeks).

A link between the IOP response of patients tested with topical corticosteroids (glucocorticoids) and the disease of POAG has long been suspected. While only 5% of the normal population shows a high IOP increase (e.g., 16 mm Hg) with topical glucocorticoid testing, greater than 40-50% of patients with POAG show this response. In addition, an Open Angle glaucoma may be induced by exposure to glucocorticoids. This observation has suggested that an increased or abnormal glucocorticoid response in trabecular cells may be involved in POAG (Zhan, G. L. et al., Exper. Eye Res. 54:211-218 (1992), the entirety of which is herein incorporated by reference; Yun, A. J. et al., Invest. Ophthamol. Vis. Sci. 30:2012-2022 (1989), the entirety of which is herein incorporated by reference; Clark, A. F., Exper. Eye Res. 55:265 (1992), the entirety of which is herein incorporated by reference; Klemetti, A., Acta Ophthamol. 68:29-33 (1990), the entirety of which is herein incorporated by reference; Knepper, P. A., U.S. Pat. No. 4,617,299, the entirety of which is herein incorporated by reference). The ability of glucocorticoids to induce a glaucoma-like condition has led to efforts to identify genes or gene products that would be induced by the cells of the trabecular meshwork in response to glucocorticoids (Polansky, J. R. et al., In: Glaucoma Update IV, Springer-Verlag, Berlin, pp. 20-29 (1991), the entirety of which is herein incorporated by reference).

Because increased IOP is a risk factor or characteristic associated with glaucoma, the measurement of this parameter by tonometry has been used as a screening procedure to help identify individuals who may have or develop visual field loss, leading to a glaucoma diagnosis (Strong, N. P., Ophthal. Physiol. Opt. 12:3-7 (1992), Greve, M. et al., Can. J. Ophthamol. 28:201-206 (1993), the entirety of which is herein incorporated by reference). However, because IOP levels may be falsely high or low, and glaucomatous and normal pressure ranges overlap, such methods are of limited value unless multiple readings are obtained (Hitchings, R. A., Br. J. Ophthamol. 77:326 (1993), the entirety of which is herein incorporated by reference; Tuck, M. W. et al., Ophthal. Physiol. Opt. 13:227-232 (1993), the entirety of which is herein incorporated by reference; Vaughan, D. et al., In: General Ophthamology, Appleton & Lange, Norwalk, Conn., pp. 213-230 (1992); Vernon, S. A., Eye 7:134-137 (1993), the entirety of which is herein incorporated by reference). The determination of elevated IOP as a risk factor for the development of glaucoma also requires testing over a period of time, because pressures may fall back into the normal range in a portion of patients evaluated. To make the diagnosis of glaucoma, examination of the optic disk and determination of the extent of a patient's visual field loss are performed (Greve, M. et al., Can. J. Ophthamol. 28:201-206 (1993)). New methods are aiding in the application of both approaches, but such tests are difficult and have their own limitations.

Novel approaches to aid in the diagnosis of a propensity for glaucoma using genetic approaches have recently been reported, involving evaluations of mutations or polymorphisms in the trabecular meshwork glucocorticoid response (TIGR), also know as the myocillin (MYOC) gene (See, e.g. Nguyen et al., U.S. Pat. Nos. 5,606,043, 5,789,169, 5,849,879, 5,854,415, 5,861,497, 6,150,161, 6,171,788, and 6,248,867; and Stone et al., U.S. Pat. Nos. 5,885,776, 5,916,778, 5,925,748, and 6,207,450, the entirety of which are herein incorporated by reference).

Non-steroidal anti-inflammatory drugs (NSAIDs) have been previously used for different ocular inflammatory conditions (using drugs that had been selected previously in systemic studies for their ability to suppress prostaglandin production and to decrease inflammatory responses in animals and humans). In the eye, NSAIDs appear to provide at least some benefit to prevent particular side-effects of surgical trauma, fluid accumulating in the back of the eye, appearance of inflammatory cells and vessel leakage in the anterior chamber, and the presence of pain.

Steroids (glucocorticoids) are reported to alleviate inflammation at least in part by inhibiting the production of prostaglandins and other eicosanoids at early stage involving the utilization of arachidonic acid (by interactions with lipomodulin-like molecules), although it is clear that other steroid actions also contribute to their anti-inflammatory effects. NSAIDs also are reported to inhibit the formation of prostaglandins and other eicosanoids at a later step (by interaction with the enzyme cyclooxygenase) as the major mechanism for their anti-inflammatory effects. However, since steroids and NSAIDs both effectively inhibit prostaglandin and other eicosanoid pathways, a concern has been that both classes of drugs might elevate IOP.

Cherng-Chyi et al., U.S. Pat. No. 5,110,493, the entirety of which is herein incorporated by reference, relates to ophthalmic non-steroidal anti-inflammatory drug formulations containing a quaternary ammonium preservative and a non-ionic surfactant. The formulations are useful for treating diseases that are either caused by, associated with or accompanied by inflammatory processes.

Doulakas, U.S. Pat. No. 4,829,088, the entirety of which is herein incorporated by reference, discloses the use of an ophthalmic medicament containing diclofenac-sodium in aqueous solution for the treatment of inflammations of the eye. Diclofenac-sodium is a non-steroidal anti-inflammatory agent which is said to be a suitable alternative for the treatment of severe acute or chronically recurrent inflammatory symptoms in the eye. The aqueous solution is made suitable for the local treatment of inflammations of the eye due to its stability against chemical decomposition of the diclofenac-sodium and preservation properties and toleration by the eye.

Nagy, U.S. Pat. No. 4,960,799, the entirety of which is herein incorporated by reference, also discloses aqueous ophthalmic solutions containing diclofenac-sodium. The solutions, having a pH of about 7.0 to about 7.8, comprise per milliliter of solution about 0.1 to about 5.0 milligrams of (a) pharmaceutically acceptable salt of ortho-(2,6-dichlophenyl-) aminophenyl acetic acid; (b) about 0.1 to about 10 milligrams of a pharmaceutically acceptable salt of ethylene diamine tetraacetic acid, (c) about 0.5 to about 200 milligrams of a pharmaceutically acceptable solubilizer, (d) about 0.01 to about 5.0 milligrams of a pharmaceutically acceptable bacteriostat and (e) the remainder water. The ophthalmic solutions are used for topical administration to the eye for the control or treatment of ocular inflammation.

### SUMMARY OF THE INVENTION

The present invention provides a method of providing treatment to a glaucomatous patient comprising (a) determining whether said patient has a mutation in the TIGR promoter selected from the group consisting of TIGRmt1, TIGRmt11, and both TIGRmt1 and TIGRmt11; and (b) administering an effective amount of a non-steroidal anti-inflammatory drug to said patient.

The present invention also provides a method of preventing increased intraocular pressure in a patient predisposed to developing glaucoma comprising (a) determining whether said patient has a mutation in the TIGR gene selected from the group consisting of TIGRmt1, TIGRmt11, and both TIGRmt1 and TIGRmt11; and (b) administering an effective amount of a non-steroidal anti-inflammatory drug to said patient.

The present invention also provides a method of determining whether an individual will show a sustained mean diurnal intraocular pressure in a patient comprising (a) determining whether said patient has a mutation in the TIGR gene selected from the group consisting of TIGRmt1, TIGRmt11, and both TIGRmt1 and TIGRmt11; and (b) administering an effective amount of a non-steroidal anti-inflammatory drug to said patient.

The present invention also provides a method for decreasing the mean diurnal intra-ocular pressure in a patient, comprising (a) identifying the presence of at least one mutation in the TIGR gene selected from the group consisting of TIGRmt1, TIGRmt11, and both TIGRmt1 and TIGRmt11; and (b) administering an effective amount of a non-steroidal anti-inflammatory drug to said patient.

The present invention also provides a method of treating a patient known to possess at least one mutation in the TIGR gene selected from the group consisting of TIGRmt1, TIGRmt11, and both TIGRmt1 and TIGRmt11 comprising (a) administering an effective amount of a non-steroidal anti-inflammatory drug to said patient; and (b) monitoring the level of intraocular pressure of said patient.

The present invention provides a method for prognosis and treatment of glaucoma in a patient comprising (a) identifying the presence of a mutation in the TIGR gene selected from the group consisting of TIGRmt1, TIGRmt11, and both TIGRmt1 and TIGRmt11; (b) administering an effective amount of a non-steroidal anti-inflammatory drug to said patient; and (c) monitoring the level of intraocular pressure in said patient.

The present invention provides a method of testing the efficacy of a therapeutic agent at counteracting glaucoma pathogenic mechanisms, comprising: a) determining whether a candidate patient for inclusion in a study has either the TIGRmt1 mutation, or both the TIGRmt1 and TIGRmt11 mutations; selecting said patient for inclusion in said study if said patient has said TIGRmt1 mutation or both of said TIGRmt1 and TIGRmt11 mutations; repeating steps a) and b) one or more times; and, testing said agent in said study.

### SUMMARY OF SEQ ID NOS

SEQ ID NO: 1 provides a nucleic acid sequence of a TIGR 5′ region from an individual without glaucoma.

SEQ ID NOs: 2-21 provide primers for DNA amplification.

### BRIEF DESCRIPTION OF THE FIGURES

FIGS. 1a, 1b, 1c, 1d, and 1e provide a nucleic acid sequence of a TIGR 5′ region (SEQ ID NO: 1).

FIG. 2 is a bar graph showing effects of TIGRmt1 on IOP in ocular hypertensives receiving a placebo treatment.

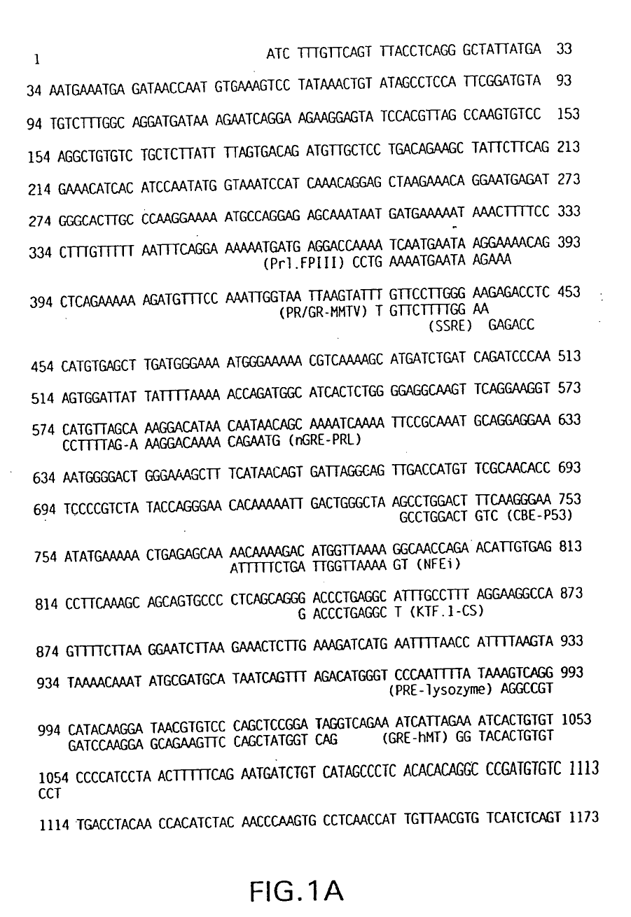

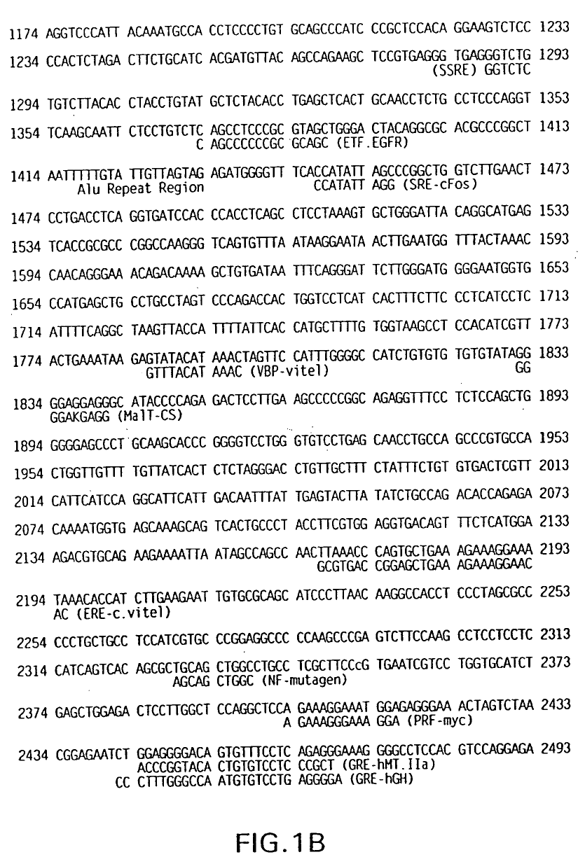

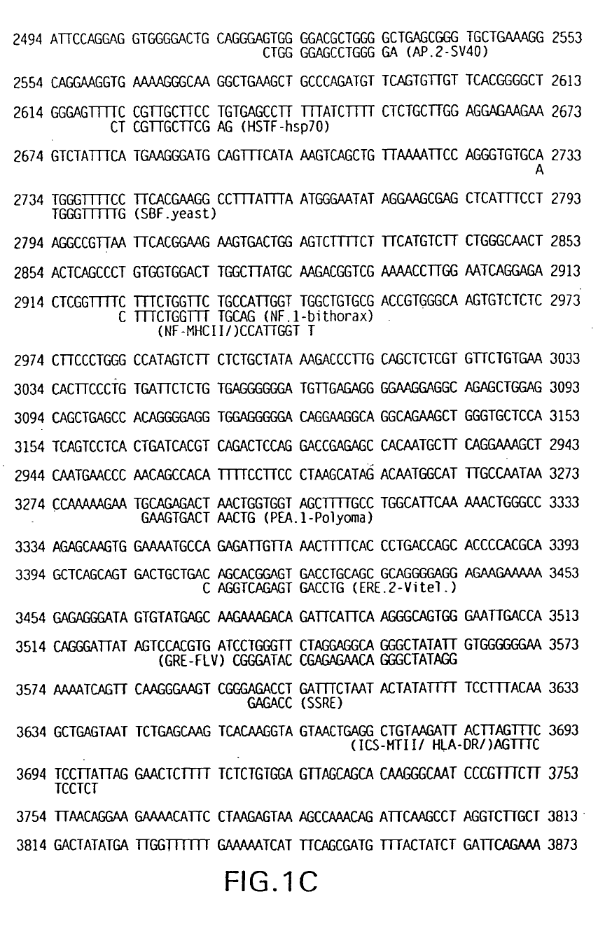

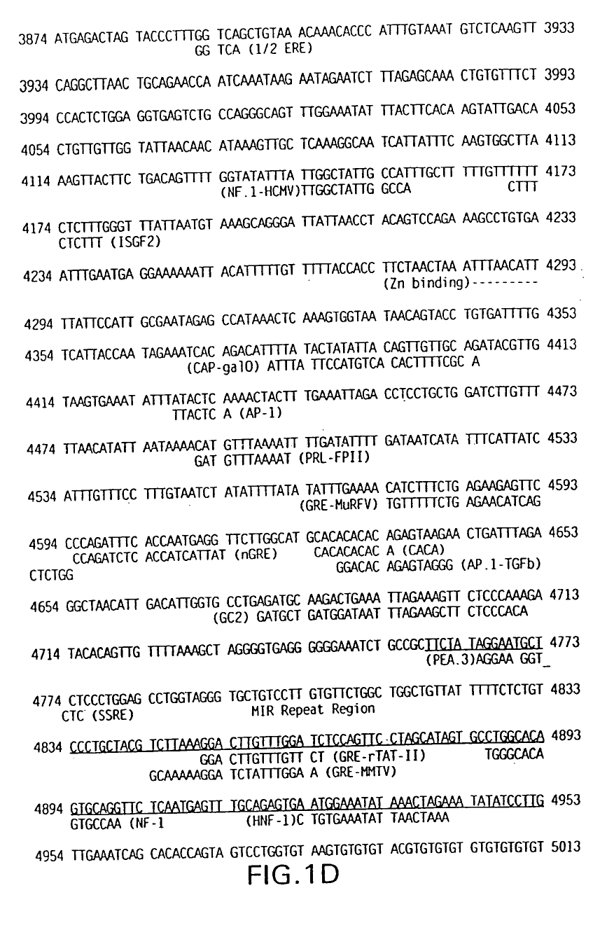

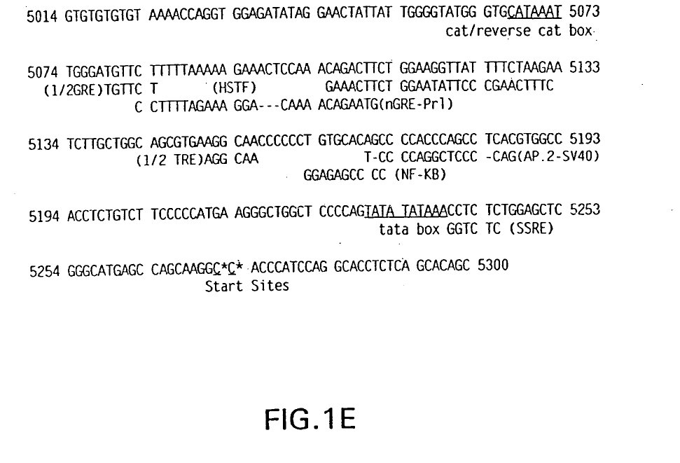

### DETAILED DESCRIPTION OF THE INVENTION

The present invention includes a method of providing treatment to a glaucomatous patient comprising (a) determining whether said patient has a mutation in the TIGR promoter selected from the group consisting of TIGRmt1, TIGRmt11, and both TIGRmt1 and TIGRmt11; and (b) administering an effective amount of a non-steroidal anti-inflammatory drug to said patient.

As used herein, the term “glaucoma” is a disease of the eye characterized by increased pressure inside the eye with resultant optic nerve damage. Glaucoma includes, but is not limited to, both primary glaucomas, secondary glaucomas, juvenile glaucomas, congenital glaucomas, and familial glaucomas, including, without limitation, pigmentary glaucoma, high tension glaucoma and low tension glaucoma and their related diseases. In one embodiment, the methods of the present invention relate to the treatment of juvenile glaucoma. In a preferred embodiment, the methods of the present invention relate to the treatment of inherited glaucomas. In a preferred embodiment, the methods of the present invention relate to the treatment of open angle glaucomas. In a more preferred embodiment, the methods of the present invention relate to the treatment of primary open angle glaucoma. In one embodiment, a disease or condition is related to glaucoma if a patient possesses or exhibits a symptom or symptoms of glaucoma. In a preferred embodiment, a disease or condition is related to glaucoma if a patient possesses or exhibits symptoms of increased IOP resulting from abnormally high aqueous outflow resistance (see, Vaughan, D. et al., In: General Ophthamology, Appleton & Lange, Norwalk, Conn., pp. 213-230 (1992)). In a more preferred embodiment, a disease or condition is related to glaucoma if a patient possesses a genotype which prognoses the development of symptoms associated with the progression of glaucoma.

As used herein, “elevated IOP” or “ocular hypertensive” refers to an IOP in an eye of a patient that is above a normal level and is correlated as a risk factor for the development of visual field loss and glaucoma.

As used herein, the term “patient” refers to any organism capable of receiving medical treatment. Preferably, a patient will be any organism recognized to be a member of the class of Mammalia. In one embodiment, patients of the present invention will be of the order primates, including humans. In a preferred embodiment, the patient will be a human.

As used herein, TIGR refers to the TIGR gene, which is also known as MYOC, Myocillin (i.e., the gene represents the one responsible for defining the “GLC1A” locus for juvenile glaucoma).

A “polymorphism” in the TIGR gene or its flanking regions is a variation or difference in the TIGR nucleic acid sequence or its flanking regions that arises in some of the members of a species. Polymorphisms are the result of a single or accumulation of mutation(s) in an unexpressed portion of a nucleic acid sequence. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e. the original “allele”) whereas other members may have the variant sequence (i.e. the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a diallelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity and paternity analysis (Weber, J. L., U.S. Pat. No. 5,075,217, the entirety of which is herein incorporated by reference; Armour, J. A. L. et al., FEBS Lett. 307:113-115 (1992), the entirety of which is herein incorporated by reference; Jones, L. et al, Eur. J. Haematol. 39:144-147 (1987), the entirety of which is herein incorporated by reference; Horn, G. T. et al., PCT Application WO91/14003, the entirety of which is herein incorporated by reference; Jeffreys, A. J., European Patent Application 370,719, the entirety of which is herein incorporated by reference; Jeffreys, A. J., U.S. Pat. No. 5,175,082, the entirety of which is herein incorporated by reference; Jeffreys. A. J. et al., Amer. J Hum. Genet. 39:11-24 (1986), the entirety of which is herein incorporated by reference; Jeffreys. A. J. et al., Nature 316:76-79 (1985), the entirety of which is herein incorporated by reference; Gray, I. C. et al., Proc. R. Acad. Soc. Lond. 243:241-253 (1991), the entirety of which is herein incorporated by reference; Moore, S. S. et al., Genomics 10:654660 (1991), the entirety of which is herein incorporated by reference; Jeffreys, A. J. et al., Anim. Genet. 18:1-15 (1987), the entirety of which is herein incorporated by reference; Hillel, J. et al., Anim. Genet. 20:145-155 (1989), the entirety of which is herein incorporated by reference; Hillel, J. et al., Genet. 124:783-789 (1990), the entirety of which is herein incorporated by reference).

In one embodiment of the present invention, a patient will be diagnosed who exhibits symptoms of glaucoma, preferably open angle glaucoma, and even more preferably primary open angle glaucoma. In one embodiment, a patient will be diagnosed who exhibits an increased IOP or steroid sensitivity.

It can be determined whether a patient has a higher propensity to develop glaucoma or a glaucoma-related condition based on methods that identify a genotype in a patient which is potentially capable of affecting the level or pattern of production of amino acids, proteins, or mRNAs associated with the TIGR gene. Methods of determining or identifying the presence of a mutation in the genetic code of an individual are known in the art. In one embodiment of the present invention, a prognosis will be given to a patient who may develop glaucoma or a related condition based on determining whether said patient possesses any mutations associated with the disease. In one embodiment, a prognosis will be given to a patient based on identifying at least one mutation in the TIGR gene, including the promoter and flanking regions. In a preferred embodiment, a prognosis will be given to a patient based on identifying the presence of TIGRmt1. In a preferred embodiment, a prognosis will be given to a patient based on identifying the presence of TIGRmt11. In another preferred embodiment, a prognosis will be given to a patient based on identifying the presence of both TIGRmt1 and TIGRmt11.

In one embodiment, identifying the presence of these mutations can be used to help identify individuals predisposed to development of juvenile glaucoma. In one embodiment, the identifying presence of these mutations can be used to help identify individuals predisposed to developing inherited glaucomas. In a preferred embodiment, identifying the presence of these mutations can be used to help identify individuals predisposed to developing open angle glaucomas. In a preferred embodiment, identifying the presence of these mutations can be used to help identify individuals predisposed to developing primary open angle glaucoma. In a preferred embodiment, identifying the presence of these mutations can be used to help identify individuals predisposed to developing steroid sensitivity. In a preferred embodiment, identifying the presence of these mutations can be used to help identify individuals predisposed to developing increased IOP.

In one embodiment, a method of detecting whether a patient has a mutation in the TIGR gene is measuring the presence of a glaucoma-causing gene product (i.e., a glaucoma causing protein, polypeptide, or peptide) in a sample obtained from a patient using standard immunoassay procedures. Polyclonal or monoclonal antibodies specific to a glaucoma-causing gene product can be generated using standard techniques.

Mutations within the TIGR gene may affect expression of the TIGR Response manifested by a cell or bodily fluid. The TIGR Response is said to be “altered” if it differs from the TIGR Response of cells or of bodily fluids of normal individuals. Such alteration may be manifested by either abnormally increased or abnormally diminished TIGR Response. To determine whether a TIGR Response is altered, the TIGR Response manifested by the cell or bodily fluid of the patient is compared with that of a similar cell sample (or bodily fluid sample) of normal individuals. As will be appreciated, it is not necessary to redetermine the TIGR Response of the cell sample (or bodily fluid sample) of normal individuals each time such a comparison is made; rather, the TIGR Response of a particular individual may be compared with previously obtained values of normal individuals.

A preferred method of detecting whether a patient has a mutation in the TIGR gene is an analysis to determine the presence and/or identity of a polymorphism in the TIGR gene, or its flanking regions which are associated with glaucoma, or a predisposition to glaucoma, related diseases, or steroid sensitivity.

The detection of polymorphic sites in a sample of DNA may be facilitated through the use of nucleic acid amplification methods. Methods for amplification, including polymerase chain reaction (“PCR”), Ligase Chain Reaction (“LCR”), Oligonucleotide Ligation Assay (“OLA”), allele-specific oligomers, branched DNA technology, transcription-based amplification systems, isothermal amplification methods, or genetic markers are well known in the art. (See generally, Mullis, K et al., Cold Spring Harbor Symp. Quant. Biol. 51:263-273 (1986), the entirety of which is herein incorporated by reference; Mullis K. et al. U.S. Pat. No. 4,683,202, the entirety of which is herein incorporated by reference; Erlich, H., U.S. Pat. No. 4,582,788, the entirety of which is herein incorporated by reference; Saiki, R. et al., U.S. Pat. No. 4,683,194, the entirety of which is herein incorporated by reference; Barany, F., Proc. Natl. Acad. Sci. (U.S.A.) 88:189-193 (1991), the entirety of which is herein incorporated by reference; Segev, D., PCT Application WO 90/01069, the entirety of which is herein incorporated by reference; Landegren, U. et al., Science 241:1077-1080 (1988), the entirety of which is herein incorporated by reference; Nickerson, D. A. et al. Proc. Natl. Acad. Sci. (U.S.A.) 87:8923-8927 (1990), the entirety of which is herein incorporated by reference; Wu, D. Y. et al., Genomics 4:560 (1989), the entirety of which is herein incorporated by reference; Malek, L. T. et al., U.S. Pat. No. 5,130,238, the entirety of which is herein incorporated by reference; Davey, C. et al., European Patent Application 329,822, the entirety of which is herein incorporated by reference; Schuster et al., U.S. Pat. No. 5,169,766, the entirety of which is herein incorporated by reference; Miller, H. I. et al., PCT appln. WO 89/06700, the entirety of which is herein incorporated by reference; Kwoh, D. et al., Proc. Natl. Acad. Sci. (U.S.A) 86:1173 (1989), the entirety of which is herein incorporated by reference; Gingeras, T. R. et al., PCT application WO 88/10315, the entirety of which is herein incorporated by reference; Walker, G. T. et al., Proc. Natl. Acad. Sci. (U.S.A.) 89:392-396 (1992), the entirety of which is herein incorporated by reference; Stone, et al., U.S. Pat. No. 5,925,748, the entirety of which is herein incorporated by reference, and Nguyen, et al., U.S. Pat. No. 6,150,161.)

Such polymorphisms can also be detected through the use of a marker nucleic acid molecule that is genetically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

Additionally, by identifying a polymorphism in the TIGR gene, it is possible to diagnose the predisposition of an asymptomatic patient to glaucoma, related diseases, or steroid sensitivity. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and animal genetic analyses (Glassberg, J., UK patent Application 2135774, the entirety of which is herein incorporated by reference; Skolnick, M. H. et al., Cytogen. Cell Genet. 32:58-67 (1982), the entirety of which is herein incorporated by reference; Botstein, D. et al., Ann. J. Hum. Genet. 32:314-331 (1980), the entirety of which is herein incorporated by reference; Fischer, S. G et al., PCT Application WO90/13668, the entirety of which is herein incorporated by reference; Uhlen, M., PCT Application WO90/11369, the entirety of which is herein incorporated by reference). Also, polymorphisms can be detected by the use of single stranded conformational polymorphism (SSCP). Orita, et al., Genomics 5:874-879 (1989), the entirety of which is herein incorporated by reference. The role of TIGR in glaucoma pathogenesis indicates that the presence of genetic alterations (e.g., DNA polymorphisms) that affect the TIGR Response can be employed to predict glaucoma.

In order to practice these methods, a sample DNA is obtained from a patient's cells. Preferably, the DNA sample is obtained from the patient's blood. However, any source of DNA may be used. The DNA is subjected to restriction endonuclease digestion. TIGR is used as a probe in accordance with the above-described RFLP methods. By comparing the RFLP pattern of the TIGR gene obtained from normal and glaucomatous patients, one can determine a patient's predisposition to glaucoma. The polymorphism obtained in this approach can then be cloned to identify the mutation at the coding region which alters the protein's structure or regulatory region of the gene which affects its expression level.

Changes involving promoter interactions with other regulatory proteins can be identified by, for example, gel shift assays using HTM cell extracts, fluid from the anterior chamber of the eye, serum, etc. Interactions of TIGR protein in glaucomatous cell extracts, fluid from the anterior chamber of the eye, serum, etc. can be compared to control samples to thereby identify changes in those properties of TIGR that relate to the pathogenesis of glaucoma. Similarly such extracts and fluids as well as others (blood, etc.) can be used to diagnosis or predict steroid sensitivity.

Several different classes of polymorphisms may be identified through such methods. Examples of such classes include: (1) polymorphisms present in the TIGR cDNA of different individuals; (2) polymorphisms in non-translated TIGR gene sequences, including the promoter or other regulatory regions of the TIGR gene; (3) polymorphisms in genes whose products interact with TIGR regulatory sequences; (4) polymorphisms in gene sequences whose products interact with the TIGR protein, or to which the TIGR protein binds.

Alternatively, the evaluation is conducted using oligonucleotide “probes” whose sequence is complementary to that of a portion of TIGR mRNA. Such molecules are then incubated with cell extracts of a patient under conditions sufficient to permit nucleic acid hybridization. For this sub-embodiment, cells of the trabecular meshwork are preferred. The detection of double-stranded probe-mRNA hybrid molecules is indicative of the presence of TIGR mRNA. The amount of such hybrid formed is proportional to the amount of TIGR mRNA. Thus, such probes may be used to ascertain the level and extent of TIGR mRNA production in a patient's cells. Such nucleic acid hybridization may be conducted under quantitative conditions (thereby providing a numerical value of the amount of TIGR mRNA present). Alternatively, the assay may be conducted as a qualitative assay that indicates either that TIGR mRNA is present, or that its level exceeds a user set, predefined value.

In other embodiments, the previously described “anti-TIGR antibodies” are employed in an immunodiagnostic assay for glaucoma and its related diseases. Methods of diagnosing glaucoma and related disorders are discussed in Nguyen, et al. U.S. Pat. No. 6,150,161 and Nguyen, et al., U.S. Pat. No. 6,171,788.

Sequences located upstream of the TIGR coding region have been isolated and sequenced in a non-glaucomic individual. The upstream sequence is set forth in SEQ ID NO: 1. Sequence comparisons of the upstream region of a non-glaucoma individual and individuals with glaucoma identify a number of mutations in individuals with glaucoma. As used herein, TIGRmt1 is a mutation of a replacement of a cytosine with a guanine at position 4337 of the TIGR promoter as shown in SEQ ID NO: 1. As used herein, TIGRmt11 is a mutation of a replacement of a thymine with a cytosine at position 5113 as shown in SEQ ID NO: 1. One or more of TIGRmt1, and TIGRmt11 can be homozygous or heterozygous.

It is a further object of the present invention to provide a method of treating a patient known to possess at least one mutation selected from the group consisting of TIGRmt1, TIGRmt11, and both TIGRmt1 and TIGRmt11 comprising (a) administering an effective amount of a non-steroidal anti-inflammatory drug to said patient and (b) monitoring the level of IOP of said patient.

Therapeutic agents of the present invention may be administered to a glaucomatous patient having a mutation selected from the group TIGRmt1 and TIGRmt11 through an appropriate vehicle or carrier and in an appropriate substantive form. In one embodiment, by formulating an NSAIDs of the present invention into an appropriate inert vehicle or carrier, it is possible to reduce or treat elevated IOP associated with steroid, corticosteroid or glucocorticoid treatment. In order to maintain an adequate therapeutic level of drug in the eye, the present invention also contemplates the treatment of an ophthalmic disease by administration of an opthalmically effective amount of the non-steroidal cyclooxygenase inhibiting agents of the present invention (including salts, hydrates, or solvates), in a suitable carrier, by oral, intramuscular and intravenous routes, in addition to the convenient topical route or by intraocular injection.

In one embodiment of the present invention, the identification of the TIGRmt1 and TIGRmt11 single nucleotide polymorphisms (SNPs), alone or in combination, will be used for pharmacogenomics and for testing of drugs to treat ocular hypertension. In a preferred embodiment, the identification of these SNPs, alone or in combination, will be used to diagnose and prevent the progression of ocular hypertension to glaucoma. In a further embodiment of the present invention, the TIGRmt1 and TIGRmt11 SNPs may be used alone or in combination with other TIGR SNPs, as well as SNPs on other genes, to test for the characteristics of ocular hypertension and glaucoma that could influence drug development and efficacy and may further provide a basis for making targeted patient management decisions, such as the frequency of monitoring IOP, optic disk, visual fields, and the like.

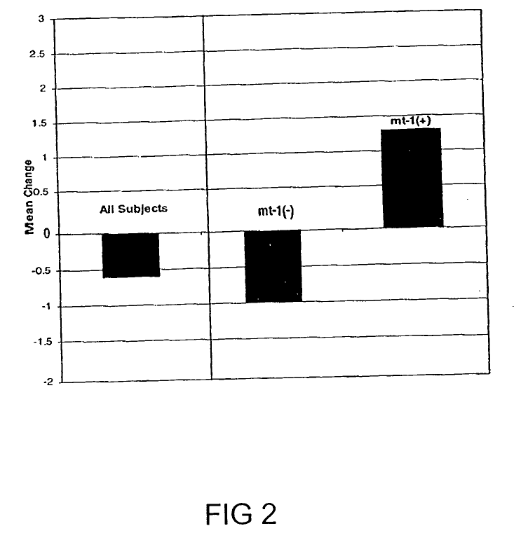

In a particularly preferred embodiment, the desirability of a patient for a study involving the use of a treatment for elevated IOP is determined by identifying whether the patient has the TIGRmt1 mutation. In a particularly preferred embodiment, the desirability of a patient for a study involving the use of a treatment for elevated IOP is determined by identifying whether the patient has the TIGRmt1 and the TIGRmt11 mutation. As shown in FIG. 2, the mean change in patients receiving a placebo treatment for elevated IOP over a six month period is significantly negative in subjects and patients lacking the TIGRmt1 mutation, but significantly positive in patients having the TIGRmt1 mutation. The mean negative change for the first two groups may be attributable to the “white coat” effect, whereby initial IOP is elevated due to the physiological stress reactions a patient may have upon encountering the IOP testing situation. As time passes, the white coat effect lessens or disappears, thus causing patients originally screened as hypertensive to no longer demonstrate elevated IOP. Patients who have TIGRmt1, however, show a mean increase in IOP over the same six month period. These results suggest that the pool of subjects selected for a clinical trial for any treatment of elevated IOP, including any treatment involving a medicament, can be greatly increased in quality by selecting candidates who have the TIGRmt1 mutation. Without such screening, a significant portion of patients can show a white coat induced decrease in IOP that is unrelated to the treatment being tested. In a preferred embodiment, patients selected based on a positive TIGRmt1 result exhibit, without treatment, an increase in IOP of greater than 0.5 mm Hg over a six month period, more preferably greater than 0.75, 1.00, 1.25, and 1.35 mm Hg over a six month period.

In another embodiment, the detection of TIGRmt1 in a patient is used to determine whether a patient has the potential to develop elevated IOP. For patients testing positive for TIGRmt1 or TIGRmt1 and TIGRmt11, treatment regimens can be introduced to prevent increases in IOP that can lead to the onset of glaucoma and related disorders. Treatment to prevent an increase in IOP over time in patients having the TIGRmt1 mutation can involve any of the treatments described herein or those otherwise known in the art, and, preferably, include either an NSAID or ISV-205. In a preferred embodiment, patients selected based on a positive TIGRmt1 and TIGRmt11 result exhibit, without treatment, an increase in IOP of greater than 0.5 mm Hg over a six month period, more preferably greater than 0.75, 1.00, 1.25, and 1.35 mm Hg over a six month period.

In a further embodiment of the present invention, in any of the embodiments disclosed herein, any one or more of the following markers can be combined, for the purpose of carrying out the method of that embodiment, with TIGRmt1, TIGRmt11, or the combination of TIGRmt1 and TIGRmt11: TIGRmt2, TIGRmt3, TIGRmt4, TIGRmt5, or TIGRsv1. That is, any of the presence or absence of the foregoing markers can be determined for the patient or subject in addition to TIGRmt1, TIGRmt11, or the combination of TIGRmt1 and TIGRmt11.

As used herein, TIGRmt2, TIGRmt3, TIGRmt4, TIGRmt5, or TIGRsv1 mean the mutations in TIGR of the same designation as disclosed in U.S. Pat. No. 6,171,788, which is herein incorporated by reference in its entirety.

In general, ophthalmic formulations suitable for topical and intraocular administration may be formulated and administered in accordance with techniques known to persons skilled in the art. The formulations are preferably prepared in an anaerobic environment by making all formulations under an inert gas. The finished formulations are preferably stored in opaque or brown containers to protect them from light exposure, and under an inert atmosphere.

Aqueous polymeric solutions, aqueous suspensions, ointments, and gels are preferably used for topical formulations. The aqueous formulations may also contain liposomes for creating a reservoir of dissolved therapeutic agent. Particularly preferred among topical formulations are gels, which enhance pre-corneal retention without the inconvenience and impairment of vision associated with ointments.

Topical ophthalmic or other topical formulations should generally include between 0.001 and 10% by weight, preferably between 0.05 and 1% by weight and even more preferably 0.05 and 0.6% by weight, of the therapeutic agent in a suitable polymeric carrier. Other preferred formulations contain between 0.001 to 0.009% by weight of the therapeutic agent. As will be appreciated by those skilled in the art, the amounts of NSAID needed to reduce IOP associated with steroid treatments include those amounts which will not effectively reduce inflammation, i.e., amounts lower than currently used in topical anti-inflammatory formulations.

Suitable polymeric carriers include lightly crosslinked carboxy-containing polymers (such as polycarbophil), dextran, cellulose derivatives, polyethyleneglycol 400 nd other polymeric demulcents.

A preferred system includes lightly crosslinked polymers of acrylic acid or the like, which are well known in the art. In a preferred embodiment, such polymers are ones prepared from at least about 90%, and preferably from about 95% to about 99.9% by weight, based on the total weight of monomers present, of one or more carboxyl-containing monoethylenically unsaturated monomers. Acrylic acid is the preferred carboxyl-containing monoethylenically unsaturated monomer, but other unsaturated, polymerizable carboxyl-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

Such polymers are crosslinked by using a small percentage, i.e., from about 0.01% to about 5%, and preferably from about 0.1% to about 2%, based on the total weight of monomers present, of a polyfunctional crosslinking agent. Included among such crosslinking agents are non-polyalkenyl polyether difunctional crosslinking monomers such as divinyl glycol; 2,3-dihydroxyhexa-1,5-diene; 2,5-dimethyl-1,5-hexadeone; divinylbenzene; N,N-diallylacrylamide; N,N-diallylmethacrylamide and the like. Also included are polyalkenyl polyether crosslinking agents containing two or more alkenyl ether groupings per molecule, preferably alkenyl ether groupings containing terminal H₂C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown, U.S. Pat. No. 2,798,053 (the entirety of which is herein incorporated by reference). Diolefinic non-hydrophilic macromeric crosslinking agents having molecular weights of from about 400 to about 8,000, such as insoluble di- and polyacrylates and methacrylates of diols and polyols, diisocyanate-hydroxyalkyl acrylate or methacrylate reaction products, and reaction products of isocyanate terminated prepolymers derived from polyester diols, polyether diols or polysiloxane diols with hydroxyalkylmethacrylates, and the like, can also be used as the crosslinking agents; see, e.g., Mueller et al., U.S. Pat. Nos. 4,192,827 (the entirety of which is herein incorporated by reference) and Mueller et al., U.S. Pat. No. 4,136,250 (the entirety of which is herein incorporated by reference).

The lightly crosslinked polymers can of course be made from a carboxyl-containing monomer or monomers as the sole monoethylenically unsaturated monomer present, together with a crosslinking agent or agents. They can also be polymers in which up to about 40%, and preferably from about 0% to about 20% by weight, of the carboxyl containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethyl-hexylacrylate, octyl methacrylate, 2-hydroxyethyl-methacrylate, 3-hydroxypropylacrylate, and the like, vinyl acetate, N-vinylpyrrolidone, and the like; see Mueller et al., U.S. Pat. No. 4,548,990 (the entirety of which is herein incorporated by reference), for a more extensive listing of such additional monoethylenically unsaturated monomers. Particularly preferred polymers are lightly crosslinked acrylic acid polymers wherein the crosslinking monomer is 2,3-dihydroxyhexa-1,5-diene or 2,3-dimethylhexa-1,5-diene.

The lightly crosslinked polymers used in practicing the present invention are preferably prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, and preferably from about 3 to about 20 μm, in equivalent spherical diameter. In general, such polymers will range in molecular weight estimated to be about 2 to 4 billion.

Aqueous suspensions formulated in accordance with the present invention containing polymer particles prepared by suspension or emulsion polymerization whose dry particle size is appreciably larger than about 50 μm in equivalent spherical diameter are less comfortable when administered to the eye than suspensions otherwise identical in composition containing polymer particles whose equivalent spherical diameters are, on the average, below about 50 μm. Lightly crosslinked polymers of acrylic acid or the like prepared to a dry particle size appreciably larger than about 50 μm in equivalent spherical diameter and then reduced in size, e.g., by mechanically milling or grinding, to a dry particle size of not more than about 50 μm in equivalent spherical diameter do not work as well as polymers made from aqueous suspensions. One possible explanation for the difference of such mechanically milled or ground polymer particles as the sole particulate polymer present is that grinding disrupts the spatial geometry or configuration of the larger than 50 μm lightly crosslinked polymer particles, perhaps by removing uncrosslinked branches from polymer chains, by producing particles having sharp edges or protrusions, or by producing ordinarily too broad a range of particle sizes to afford satisfactory delivery system performance. A broad distribution of particle sizes will impair the viscosity-gelation relationship. In any event, such mechanically reduced particles are less easily hydratable in aqueous suspension than particles prepared to the appropriate size by suspension or emulsion polymerization, and also are less able to gel in the eye under the influence of tear fluid to a sufficient extent and are less comfortable once gelled than gels produced in the eye using the aqueous suspensions of the present invention. However, up to about 40% by weight, e.g., from about 0% to over 20% by weight, based on the total weight of lightly crosslinked particles present, of such milled or ground polymer particles can be admixed with solution or emulsion polymerized polymer particles having dry particle diameters of not more than about 50 μm when practicing the present invention. Such mixtures will also provide satisfactory viscosity levels in the ophthalmic medicament delivery systems with ease and comfort of administration and satisfactory sustained release of the medicament to the eye, particularly when such milled or ground polymer particles, in dry form, average from about 0.01 to about 30 μm, and preferably from about 1 to about 10 μm, in equivalent spherical diameter.

In the most preferred embodiment of the invention, the particles have a narrow particle size distribution. The use of a monodisperse particle will give maximum viscosity and an increased eye residence time of the ophthalmic medicament delivery systems for a given particle size. Monodisperse particles having a particle size of 30 μm and below are most preferred. Good particle packing is aided by a narrow particle size distribution.

The particles are not only affected by the upper size limits described above, but also to a narrow particle size distribution. Such use of a monodispersion of particles, which aids in good particle packing, yields a maximum increased viscosity upon contact of the suspension with the tears and increases eye residence time. At least about 80%, more preferably at least about 90% and most preferably at least about 95%, of the particles should be within a no more than about 10 μm band of major particle size distribution, and overall (i.e., considering particles both within and outside such band) there should be no more than about 20%, preferably no more than about 10% and most preferably no more than about 5% fines (i.e., particles of a size below 1 μm). It is also preferred that as the average particle size is lowered from the upper limit of 50 μm, more preferably 30 μm, to lower sizes such as 6 μm, that the band of major particle distribution be also narrowed, for example to 5 μm. Preferred sizes for particles within the band of major particle distribution are less than about 30 μm, more preferably less than about 20 μm, most preferably from about 1 μm to about 5 μm.

The aqueous suspensions of this invention may preferably contain amounts of lightly crosslinked polymer particles ranging from about 0.1% to about 6.5% by weight, and preferably from about 0.5% to about 4.5% by weight, based on the total weight of the aqueous suspension. They will preferably be prepared using pure, sterile water, preferably deionized or distilled, having no physiologically or ophthalmologically harmful constituents, and will be adjusted to a neutral pH of about 7.0 to about 7.4 using any physiologically and ophthalmologically acceptable pH adjusting acids, bases or buffers, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylamino-methane), or the like and salts and buffers such as citrate/dextrose, sodium bicarbonate, ammonium chloride and mixtures of the aforementioned acids and bases.

When formulating the aqueous suspensions of the present invention, their osmotic pressure (π) will be adjusted to from about 10 milliosmolar (mOsM) to about 400 mOsM, and preferably from about 100 to about 250 mOsM, using appropriate amounts of physiologically and ophthalmologically acceptable salts. Sodium chloride is preferred to approximate physiologic fluid, and amounts of sodium chloride ranging from about 0.01% to about 1% by weight and preferably from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The amounts of lightly crosslinked polymer particles, the pH, and the osmotic pressure chosen from within the above-stated ranges will be correlated to give aqueous suspensions preferably having viscosities ranging from about 1,000 to about 30,000 centipoise, and preferably from about 5,000 to about 30,000 centipoise, as measured at room temperature (about 25° C.) using a Brookfield Digital LVT Viscometer equipped with a number 25 spindle and a 13R small sample adapter at 12 rpm. Higher viscosities may also be employed, and formulations of less than 100,000 centipoise can be administered as a ribbon.

The viscous gels that result from fluid eyedrops delivered by means of the aqueous suspensions of this invention have residence times in the eye ranging from about 2 to about 12 hours, e.g., from about 3 to about 6 hours. The medicaments contained in these drug delivery systems will be released from the gels at rates that depend on such factors as the drug itself and its physical form, the extent of drug loading and the pH of the system, as well as on any drug delivery adjuvants, such as ion exchange resins compatible with the ocular surface, which may also be present. Preferably, the aqueous suspensions provide a sustained concentration of cyclooxygenase inhibitor of between 10⁻⁸ and 10⁻⁶ M, and more preferably between 10⁻⁷ and 10⁻⁵ M, in the aqueous or treated tissue of the eye for at least two hours, preferably at least three hours.

The aqueous suspension topical ophthalmic medicament delivery systems of the present invention can be formulated in any of several preserved or nonpreserved ways. For example the drug, the lightly crosslinked polymer particles, and the osmolality-adjusting salt can be pre-blended in dry form, added to all or part of the water, and stirred vigorously until apparent polymer dispersion is complete, as evidenced by the absence of visible polymer aggregates. Sufficient pH adjusting agent is then added incrementally to reach the desired pH, and more water to reach 100 percent formula weight can be added at this time, if necessary. Another convenient method involves adding the drug to about 95 percent of the final water volume and stirring for a sufficient time to saturate the solution. Solution saturation can be determined in known manner, e.g., using a spectrophotometer. The lightly crosslinked polymer particles and the osmolality-adjusting salt are first blended in dry form and then added to the drug-saturated suspension and stirred until apparent polymer hydration is complete. Following the incremental addition of sufficient pH adjusting agent to reach the desired pH, the remainder of the water is added, with stirring, to bring the suspension to 100 percent formula weight.

These aqueous suspensions can be packaged in preservative-free, single-dose non-reclosable containers. This permits a single dose of the medicament to be delivered to the eye one drop at a time, with the container then being discarded after use. Such containers eliminate the potential for preservative-related irritation and sensitization of the corneal epithelium, as has been observed to occur particularly from ophthalmic medicaments containing mercurial preservatives. Multiple-dose containers can also be used, if desired, particularly since the relatively low viscosities of the aqueous suspensions of this invention permit constant, accurate dosages to be administered dropwise to the eye as many times each day as necessary. In those suspensions where preservatives are to be included, suitable preservatives are chlorobutanol, Polyquat, benzalkonium chloride, cetyl bromide, and the like.

Other additives which are desirably included in the topical formulations include sodium chloride, EDTA (disodium edetate), surfactants, and preservatives like BAK (benzalkonium chloride). Administration of the formulation to the eye will typically be carried out between one and four times a day, depending on the particular problem being treated.

Formulations suitable for ocular injection fall into two categories. For subconjunctival injection, the formulations should generally include between 0.001 and 5% by weight, preferably between 0.01 and 1% by weight of therapeutic agent. Any suitable carriers may be employed, preferably polymeric carriers such as dextran or polysorbate 80. Other additives which desirably may be included in the formulations are disodium edetate and sodium sulfite. To administer the formulations to the eye, the drug formulations will be slowly injected into the bulbar conjunctiva of the eye.

For intracameral or intravitreal injections, the suitable formulation should include phosphate buffered saline, citrate buffered saline, chondroitin sulfate, or a polymeric carrier such as sodium hyaluronate (or hyaluronic acid), purified polyacrylamide or polysorbate 80. Other additives which are desirably included in the ocularly injectable formulations are sodium chloride, sodium hydroxide and hydrogen chloride, where sodium hydroxide and hydrogen chloride are used for adjustment of pH. Typically, the formulations contain between 0.001 and 1%, preferably between 0.01 and 1.0% especially when in solution, by weight of the agent.

When the agent is substantially in solution, it is rapidly available to exert its therapeutic function and lower concentrations may therefore be administered to achieve effective levels without causing tissue intolerance. When the agent is substantially in suspension, higher concentrations may be administered to achieve a sustained effective level, again without causing tissue intolerance. Hence, with solutions, lower concentrations are employed to avoid local tissue damage. With a suspension, higher concentrations are employed because a smaller dissolved amount is introduced for immediate activity.

To administer the formulations intravitreally to the eye, the drug formulation will be injected through the sclera layer of the eye into the vitreous cavity. To administer the formulations intracamerally, the drug formulations will be injected through the cornea into the anterior chamber of the eye.

Formulations for intravenous, intramuscular, and oral administration are likewise prepared in accordance with techniques well known to persons skilled in the art. Intravenous formulations for ophthalmic use in methods of the present invention may be prior art formulations used for other purposes and will typically include between 0.01 and 50.0% by weight and preferably between 1.0 and 10.0% by weight of the therapeutic agent. Suitable carriers for such NSAIDs are those well known to persons skilled in the art such as citrate buffer, borate buffer and others. Other additives which may be desirably added to intravenous formulations include sodium chloride, sodium sulfite, disodium edetate and benzyl alcohol. Alternative formulations suitable for intravenous administration include carriers such as lipid emulsions containing the therapeutic agent. To administer the intravenous formulations for treatment of the eye, the drug formulations are preferably dose injected or infused into a major vein (e.g., in the arm area), or introduced by continuous intravenous drip.

Intramuscular formulations will typically include between 0.01 and 10.0% by weight and preferably between 0.5 and 5.0% by weight of the therapeutic agent. Suitable adjuvants in aqueous solution or suspension for intramuscular lazaroid formulations are those well known to persons skilled in the art such as polysorbate 80, methyl cellulose, and other demulcents. Other additives desirably added to intramuscular formulations include sodium chloride and sodium bisulfite. To administer the intramuscular formulations for treatment of the eye, the drug formulations will be injected for example into the upper outer quadrant of the gluteal muscle.

Formulations suitable for oral administration will include both liquid formulations (aqueous solutions, aqueous suspension, elixirs, and the like) and solid dosage forms, both containing additives and adjuvants well known to persons skilled in the art. Aqueous solutions and suspensions for liquid oral administration will typically contain between 0.05 and 50% by weight and preferably between 1.0 and 10.0% by weight of the NSAID. Suitable adjuvants may be used as carriers to provide wetability and stability such as propylene glycol, lightly crosslinked carboxy-containing polymers such as polycarbophil, ethyl cellulose, hydroxypropyl cellulose and methyl cellulose. Other additives, including sodium edetate, methyl and propyl parabens, flavoring agents and colorants may be employed if desirable. Solid dosage forms for oral administration may also be prepared as capsules, caplets or tablets with the aid of fillers, lubricants and stabilizers. To administer oral formulations for treatment of the eye, the drug is swallowed in solid dosage form or as a solution or suspension.

An “effective amount” of the agents administered in the present invention is the pharmacological amount of a selected agent to be medically effective in the treatment of glaucoma or glaucoma-related conditions. In one embodiment, the effective amount of the selected agent will the amount required to sustain the IOP (IOP) in the eye of a glaucomatous patient. In a preferred embodiment, the effective amount will be the amount required to reduce the IOP in the eye of a glaucomatous patient. In a preferred embodiment, the agent will be an NSAID or non-steroidal cyclooxygenase inhibitor.

The precise amount of an NSAID or non-steroidal cyclooxygenase inhibitor for use in the present compositions will vary as well. Generally, an effective amount of NSAID or related compound will be the amount required to prevent an increase in, or sustain the current level of IOP in a patient for at least six months. In one embodiment, the level of IOP will be sustained at about 22 mm Hg for at least six months. In a preferred embodiment, an effective amount will decrease the level of IOP associated with steroid, corticosteroid or glucocorticoid treatment for at least six months.

In one embodiment, an effective amount is a composition comprising about 0.01% by weight of an NSAID. In a preferred embodiment, an effective amount is a composition comprising about 0.03% by weight of an NSAID. In a more preferred embodiment, an effective amount is a composition comprising about 0.06% by weight of an NSAID. In an even more preferred embodiment, an effective amount is a composition comprising about 0.1% by weight of an NSAID.

NSAIDs are widely prescribed to reduce pain and inflammation in a wide number of tissues. This includes their application as topical agents in the eye, in which their ability to suppress inflammatory responses and to prevent particular side-effects of surgical trauma (on the pupil preventing surgical meiosis), fluid accumulating in the back of the eye after cataract surgery (post-surgical macular edema) and the appearance of inflammatory cells and vessel leakage in the anterior chamber. Topical application of NSAIDs in the eye also appears to relieve some of the itching due to allergic conjunctivitis. These conditions fit in the normal and expected effects of NSAIDs in inflammation and pain.

“Cyclooxygenase inhibiting agents” include those compounds which inhibit prostaglandin and other eicosanoid or cyclooxygenase pathways which are reportd to affect IOP. Compounds considered within the classification of cyclooxygenase inhibitors include certain NSAIDs.

NSAIDs have been documented by J. Lombardino, In: Nonsteroidal Antinflammatory Drugs, Wiley-Interscience, New York, 1985, the entirety of which is herein incorporated by reference. In one embodiment, examples of compounds of this class of anti-inflammatory drugs include but are not limited to the following: aspirin, benoxaprofen, benzofenac, bucloxic acid, butibufen, carprofen, cicloprofen, cinmetacin, clidanac, clopirac, diclofenac, etodolac, fenbufen, fenclofenac, fenclorac, fenoprofen, fentiazac, flunoxaprofen, furaprofen, flurbiprofen, furobufen, furofenac, ibuprofen, ibufenac, indomethacin, indoprofen, isoxepac, ketoprofen, lactorolac, lonazolac, metiazinic, miroprofen, naproxen, oxaprozin, oxepinac, phenacetin, pirprofen, pirazolac, protizinic acid, sulindac, suprofen, tiaprofenic acid, tolmetin, and zomepirac.

Non-steroidal cyclooxygenase inhibiting compounds can be prepared in the form of pharmaceutically acceptable salts, esters and other prodrugs. Derivative salts include relatively non-toxic inorganic or organic acid addition salts or alkaline earth metal salts of the therapeutic compounds, which can be prepared in situ during the final isolation and purification of the compounds or by separately reacting the free base with a suitable organic or inorganic acid. Where the compounds include a basic functionality such as amine or alkylamine, representative salts include hydrochloride, sulfate, acetate, maleate, lauryl sulphate, and the like. Where an acidic functionality is present, salts such as sodium, calcium, potassium and magnesium salts may be formed.

While some NSAIDs are primarily used at the present time as anti-inflammatory agents and others are primarily used as analgesics, in fact it is believed that all of the contemplated compounds have both analgesic and anti-inflammatory activity and can be used at appropriate dosage levels for either purpose in various compositions.

A non-steroidal anti-inflammatory drug (NSAID) is preferably a member of the class of compounds that represent useful therapeutic agents for treating certain chronic, non-inflammatory forms of glaucoma, preferably for treatment of open angle glaucomas and even more preferably for treatment of primary open angle glaucoma. In a preferred embodiment, the phrase “NSAID” as used herein is intended to mean any non-narcotic analgesic/non-steroidal anti-inflammatory compound useful as a cyclooxygenase inhibitor, including but not limited to the derivatives of (1) propionic acid, (2) acetic acid derivatives, (3) fenamic acid, (4) biphenylcarboxylic acid and (5) oxicams. In a more preferred embodiment, an NSAID will be selected from the group of cyclooxygenase inhibitors. In an even more preferred embodiment, the cyclooxygenase inhibitor will be selected from the group of phenylacetic acids.

The compounds in groups (1) through (4), listed above, typically contain a carboxylic acid function; however, those acids are sometimes administered in the form of their pharmaceutically acceptable acid addition or alkali metal salts, e.g., sodium salts.

The propionic acid derivatives include, but are not limited to, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alimoprofen, tiaprofenic acid, fluprofen and bucloxic acid. Structurally related propionic acid derivatives having similar analgesic and anti-inflammatory properties are also intended to be encompassed by this group.

Thus, “propionic acid derivatives” as defined herein are non-narcotic analgesics/non-steroidal anti-inflammatory drugs having a free —CH(CH₃)COOH or —CH₂CH₂COOH group (which optionally can be in the form of a pharmaceutically acceptable salt group, e.g., —CH(CH₃)COO⁻Na⁺), typically attached directly or via a carbonyl function to a ring system, preferably to an aromatic ring system.

The acetic acid derivatives as defined herein include, but are not limited to, indomethacin, sulindac, tolmetin, zomepirac, diclofenac, fenclofenac, alclofenac, ibufenac, isoxepac, furofenac, tiopinac, zidometacin, acemetacin, fentiazac, clidanac and oxpinac. Structurally related acetic acid derivatives having similar analgesic and anti-inflammatory properties are also intended to be encompassed by this group. Most preferably, this group includes phenylacetic acids.

Thus, “acetic acid derivatives” as defined herein are non-narcotic analgesics/non-steroidal anti-inflammatory drugs having a free —CH₂COOH group (which optionally can be in the form of a pharmaceutically acceptable salt group, e.g., —CH₂COO⁻Na⁺), typically attached directly to a ring system, preferably to an aromatic or heteroaromatic ring system.

The fenamic acid derivatives as defined herein include, but are not limited to, mefenamic acid, meclofenamic acid, flufenamic acid, niflumic acid and tolfenamic acid. Structurally related fenamic acid derivatives having similar analgesic and anti-inflammatory properties are also intended to be encompassed by this group.

Thus, “fenamic acid derivatives” as defined herein are non-narcotic analgesics/non-steroidal anti-inflammatory drugs which contain the basic structure

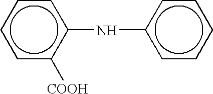

which can bear a variety of substituents and in which the free —COOH group can be in the form of a pharmaceutically acceptable salt group, e.g., —COO⁻ NA⁺.

The biphenylcarboxylic acid derivatives as defined herein include, but are not limited to, diflunisal and flufenisal. Structurally related biphenylcarboxylic acid derivatives having similar analgesic and anti-inflammatory properties are also intended to be encompassed by this group.

Thus, “biphenylcarboxylic acid derivatives” as defined herein are non-narcotic analgesics/non-steroidal anti-inflammatory drugs which contain the basic structure

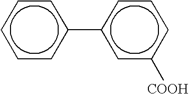

which can bear a variety of substituents and in which the free —COOH group can be in the form of a pharmaceutically acceptable salt group, e.g., —COO⁻NA⁺

The oxicams as defined herein include, but are not limited to, piroxicam, sudoxicam, isoxicam, and CP-14,304. Structurally related oxicams having similar analgesic and anti-inflammatory properties are also intended to be encompassed by this group. A preferred member of this group is piroxicam.

Thus, “oxicams” as defined herein are non-narcotic analgesics/non-steroidal anti-inflammatory drugs which have the general formula:

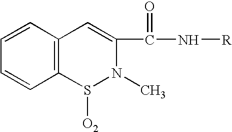

wherein R is an aryl or heteroaryl ring system.

The preferred NSAIDs of the present invention also include the non-steroidal cyclooxygenase inhibitors as described by Flach, In: Cyclooxygenase Inhibitors in Ophthalmology, Survey of Ophthalmology; Vol. 36, No. 4, (Jan.-Feb. 1992), the entirety of which is herein incorporated by reference. Cyclooxygenase inhibitors are also non-steroidal anti-inflammatory drugs that have become available as ophthalmic eyedrops for treatment of inflammation. These inhibitors may be grouped into six different classes: salicylates, fenamates, indoles, phenylalkanoic acids and pyrazolones. Specific drugs within the respective groups are summarized below.

Cyclooxygenase Inhibitors

| Chemical Class | Generic Name |
|---|---|
| Salicylates | Aspirin, Salicylic Acid, |
| | Diflunisol |
| Indoles | Indomethacin, Sulinda, |
| | Tolmetin |
| Phenylalkanoic acids | Fenoprofen, Flurbiprofen, |
| | Ibuprofen, Ketoprofen, |
| | Ketorolac, Naproxen, |
| | Piroxicam, Suprofen |
| Phenylacetic acids | Diclofenac |
| Pyrazolons | Oxyphenbutazone, |
| | Phenylbutazone, Antipyrine, |
| | Aminopyrine, Azapropazone |

The precise type of NSAID or non-steroidal cyclooxygenase or other eicosanoid inhibitor for use in the present compositions will vary depending, for example, on the specific drug chosen, the dosage form thereof, i.e., standard versus sustained release, the condition for which the drug is administered and the size and kind of the organism treated. In a preferred embodiment, the NSAID is selected from the group of non-steroidal cyclooxyganase inhibitors. In a preferred embodiment, the non-steroidal cyclooxyganase inhibitor is selected from the group of phenylacetic acids. In a more preferred embodiment, diclofenac is selected for administration to a glaucomatous patient.

It is within the ability of those skilled in the art to determine, upon reading this disclosure, which of the foregoing cyclooxygenase inhibiting agents will function to prevent an increase of IOP or decrease IOP associated with steroid, corticosteroid or glucocorticoid treatment. Preferably, those compounds of the present invention include all non-steroidal cyclooxygenase inhibitors which provide a reduction in or prevention of enhanced IOP induced by glucocorticoid treatment when used in an amount sufficient to provide a concentration of 1×10⁻⁵ M or less, preferably an amount of about 1×10⁻⁹ M to about 1×10⁻⁵ M, more preferably about 1×10⁻⁸ M to about 1×10⁻⁶ M in the aqueous or treated tissue of the eye.

It is also an object of the present invention to provide a method of preventing increased IOP in a patient predisposed to developing glaucoma comprising (a) determining whether said patient possesses a mutation selected from the group consisting of TIGRmt1, TIGRmt11, and both TIGRmt1 and TIGRmt11 and (b) administering an effective amount of a non-steroidal anti-inflammatory drug to said patient.

In one embodiment, an NSAID is administered to a symptomatic glaucomatous patient. In a preferred embodiment, preventing increased IOP is based on anticipating the likely development of glaucoma or a glaucoma-related condition by identification of the disclosed mutations in the TIGR promoter. Hence, in a preferred embodiment, intervention using the disclosed methods will take place in an asymptomatic patient, i.e., a patient that exhibits no obvious symptoms of having glaucoma.

It is a further object of the present invention to provide a method of sustaining the mean diurnal IOP in a patient comprising (a) determining whether said patient has a mutation selected from the group consisting of TIGRmt1, TIGRmt11, and both TIGRmt1 and TIGRmt11 and (b) administering an effective amount of a non-steroidal anti-inflammatory drug to said patient.

Studies of human trabecular meshwork (HTM) cells grown in tissue culture in which biochemical and morphological responses have been evaluated and have provided a model system to evaluate the mechanisms for the development of steroid effects to raise IOP, and provide a means to investigate new therapeutic approaches. Under appropriate cell culture conditions, HTM cells may be propagated using sufficiently high split ratios to obtain populations of these cells at early passages for reproducible experimental evaluations. In vitro studies of confluent, stable monolayers of HTM cells reveal a variety of structural and functional properties of the trabecular meshwork cell type which appear important for normal maintenance of the aqueous humor outflow pathway. Using these cells, it is possible to consider alterations produced by steroids and other drugs that may be related to effects on IOP.

Investigations of HTM cells with topical glucocorticoid treatment proved that steroids such as dexamethasone produced major new protein inductions in HTM cells which became progressively more noticeable between 1 and 3 weeks of 100 nM dexamethasone exposure. The correlation between dexamethasone effects on these protein inductions and the clinically observed rise in IOP suggested that prolonged glucocorticoid treatments on HTM cells provide a model system to study steroid effects on outflow facility, as described in Polansky et al., “Glucocorticoid regulation of cultured human trabecular cells: a model system to study effects of steroids on IOP”, Invest Ophthalmol Vis Sci 26:5 (1985). Studies of the HTM model system have reported that inductions of protein/glycoproteins in the molecular weight range of about 54-56 kDa (glucocorticoid-induced protein at 55 kDa, termed GIP-55 in the above-cited publication) and about 65-67 kDa (glucocorticoid-induced protein at 66 kDa, termed GIP-66 in the above-cited publication) were found in cytosol and media fractions, respectively, of dexamethasone treated HTM cultures. Thus, these proteins/glycoproteins provide a suitable marker for steroid induced elevated IOP since these inductions were not observed in the non-treated controls evaluated in this study. As mentioned above, these markers are referred to herein as 55 kDa and 66 kDa protein/glycoprotein marker inductions but, of course, as will be appreciated by those skilled in the art, the actual molecular weight of the protein according to the methods described herein, i.e., gel electrophoresis, is within a range of the recited values and when referred to herein such marker induction should include the major induction within the range.

Using the above models, practicing the methods of the present invention unexpectedly found that NSAIDs, and preferably the non-steroidal cyclooxygenase inhibiting agents, do not induce, or induce to a minimal extent, the protein markers for elevated IOP in the model system. In fact, it has been discovered that conjoint treatment of steroids and NSAIDs provides protein/glycoprotein marker reduction and, thus, would be expected to help minimize or prevent the elevated IOP found with steroid treatment.

In one embodiment of the present invention, the level of IOP in a patient treated with the disclosed methods will be sustained at the level identified at the onset of glaucoma symptoms for at least six months. In a preferred embodiment, the level of IOP in a patient will be about 25 mm Hg per six months. In a more preferred embodiment, the level of IOP in a patient will be about 22 mm Hg per six months. In a most preferred embodiment, the level of IOP in a patient will be about 20 mm Hg per six months.

The present invention also provides a method for decreasing the mean diurnal IOP in a patient comprising (a) identifying the presence of at least one mutation selected from the group TIGRmt1, TIGRmt11, and both TIGRmt1 and TIGRmt11, and (b) administering an effective amount of a non-steroidal anti-inflammatory drug to said patient.

According to preferred embodiments of the invention, the cyclooxygenase inhibiting agents or NSAIDs of the present invention provide a protein/glycoprotein marker reduction of steroid induced glycoprotein markers, i.e., either 55 kDA or 66 kDa proteins, where the marker reduction equals

$$\frac{\begin{matrix}induction of glyco \text{-} \\ protein with steroid \\ treatment alone \end{matrix} - \begin{matrix}induction of glycoprotein \\ with combination of steroid \\ and non \text{-} steroid treatment \end{matrix}}{induction of glycoprotein \\ with steroid treatment alone} \times 100 \%$$

Of course, steroid induced elevated IOP may also be subsequently treated using the non-steroidal cyclooxygenase agents of the present invention. In such case, the agent may be applied to reduce elevated IOP. In this case, the marker reduction equals

$$\frac{\begin{matrix}induction of glycoprotein \\ with steroid \\ treatment alone \end{matrix} - \begin{matrix} induction of glycoprotein \\ after treatment with non \text{-} \\ steroid al agent \end{matrix}}{induction of glycoprotein \\ with steroid treatment alone} \times 100 \%$$

Most preferably, the non-steroidal inhibiting agents or NSAIDs used in the methods and compositions of the present invention are used in an amount sufficient to provide a protein marker reduction of at least about 5%, more preferably at least about 10%, even more preferably at least about 20% and most preferably at least about 40%. Of course, the amount of protein marker reduction depends on the type and amount of non-steroidal cyclooxygenase inhibiting agent or NSAID used.

In view of the above, the non-steroidal cyclooxygenase inhibiting agents or NSAIDs used in the methods and compositions of the present invention are used in an amount sufficient to provide reduced IOP by at least about 5%, more preferably at least about 10%, even more preferably at least about 20%, and even more preferably at least about 40%. Again, however, the amount of IOP reduction depends on the amount and type of non-steroidal cyclooxygenase inhibiting agent or NSAI agent used.

In one embodiment of the present invention, the decrease in mean diurnal IOP is greater than about 1 mm Hg per six months. In a preferred embodiment of the present invention, the decrease in mean diurnal IOP is greater than about 1.5 mm Hg per six months. In a more preferred embodiment, the decrease in mean diurnal IOP is greater than about 2.0 mm Hg per six months. In an even more preferred embodiment, the decrease in mean diurnal IOP is greater than about 2.5 mm Hg per six months. In a most preferred embodiment, the decrease in mean diurnal IOP is greater than about 3.0 mm Hg per six months.

The precise amount of an anti-inflammatory drug for use will vary depending on the specific condition for which the drug is administered and for the size and type of patient treated. It is within the ability of those skilled in the art to determine, upon reading this disclosure, what will constitute and effective amount under the particular conditions present.

It is a further object of the present invention to provide a method for prognosis and treatment of glaucoma in a patient comprising (a) identifying the presence of at least one mutation selected from the group consisting of TIGRmt1, TIGRmt11, and both TIGRmt1 and TIGRmt11, (b) administering an effective amount of a non-steroidal anti-inflammatory drug to said patient, and (c) monitoring the level of IOP in said patient.

In one embodiment, the methods of the present invention will disclose a prognosis at the earliest onset of symptoms of glaucoma or a glaucoma-related condition. In a preferred embodiment, a prognosis will be made when a patient having a propensity to develop glaucoma who has not yet exhibited symptoms. In the most preferred embodiment of the present invention, the methods provided will be used to determine a prognosis for the development of glaucoma and prevent increased IOP in a patient not yet exhibiting symptoms.

In a further embodiment, the present invention includes a method of testing the efficacy of a therapeutic agent at counteracting glaucoma pathogenic mechanisms, comprising: a) determining whether a candidate patient for inclusion in a study has either the TIGRmt1 mutation, or both the TIGRmt1 and TIGRmt11 mutations; b) selecting said patient for inclusion in said study if said patient has said TIGRmt1 mutation or both of said TIGRmt1 and TIGRmt11 mutations; c) repeating steps a) and b) one or more times; and, d) testing said agent in said study.

As used herein, a “testing an agent in a study” means administering the agent in any suitable form to patients in a patient pool and observing the patients to determine whether symptoms or characteristics associated with the pathogenic mechanisms responsible for glaucoma have been affected in a manner that indicates that the detrimental effects of the pathogenic mechanisms have been either ameliorated or eliminated. As used herein, a “glaucoma pathogenic mechanism” is any biological mechanism that is, by itself or in combination with other mechanisms, causative of glaucoma.

In a preferred embodiment, the agent comprises a member selected from the group consisting of prostaglandin inhibitors, selective or non-selective COX 1 or COX 2 inhibitors, antioxidants, neuroprotective agents, platelet activating factor antagonists, alpha agonists, beta blockers, beta agonists, prostaglandin agonists, hypotensive lipids, carbonic anhydrase inhibitors, cholinergic agents, and combinations of the foregoing, including prodrugs and modified versions of the foregoing, and those in the specific embodiments listed below. In another embodiment, the agent comprises a health food supplement such as ginko biloba.

In another embodiment, the agent comprises an alpha agonist. In a preferred embodiment, the alpha agonist comprises clonidine, apoclonidine, or bromonidine, or combinations of the foregoing.

In another embodiment, the agent comprises a beta blocker. In a preferred embodiment, the beta blocker comprises timolol, betaxolol, meteripanolol or carteolol, or combinations of the foregoing.

In another embodiment, the agent comprises a beta agonist. In a preferred embodiment, the beta agonist comprises epinephrine or isoprotanerol, or a combination of both.

In another embodiment, the agent comprises a prostaglandin agonist. In a preferred embodiment, the prostaglandin agonist comprises PGF2 alpha or travoprost, or a combination of both.

In another embodiment, the agent comprises a hypotensive lipid. In a preferred embodiment, the hypotensive lipid comprises bimatoprost.

In another embodiment, the agent comprises a carbonic anhydrase inhibitor. In a preferred embodiment, the carbonic anhydrase inhibitor comprises dorzolamide HCl.

In another embodiment, the agent comprises a cholinergic agent. In a preferred embodiment, the cholinergic agent comprises pilocarpine, carbachol, or a combination of both.

The methods disclosed herein are better understood in light of and with reference to the following examples. The examples are illustrative only and are not used in a limiting sense.

### EXAMPLE I

### Identification of Mutations in the TIGR Gene

Patient samples are initially screened by Single Stranded Conformational Polymorphism (SSCP) using the method of Hue et al., The Journal of Investigative Ophthalmology 105.4: 529-632 (1995), herein incorporated by reference in its entirety. Samples, whose migration pattern indicated the potential presence of a polymorphism are sequenced and the sequence by the method of Sanger et al. J. Mol. Biol. 25;94(3):441-8 (1975), which is herein incorporated by reference in its entirety.

Single strand conformational polymorphism (SSCP) screening is carried out according to the procedure of Hue et al., The Journal of Investigative Ophthalmology 105.4: 529-632 (1995), herein incorporated by reference. SSCP primers are constructed corresponding to sequences found within the TIGR promoter and two of exons of TIGR. The following primers are constructed: forward primer “Sk-1a”: 5′-TGA GGC TTC CTC TGG AAA C-3′ (SEQ ID NO: 2); reverse primer “ca2”: 5′-TGA AAT CAG CAC ACC AGT AG-3′ (SEQ ID NO: 3); forward primer “CA2”: 5′-GCA CCC ATA CCC CAA TAA TAG-3′ (SEQ ID NO: 4); reverse primer “Pr+1”: 5′-AGA GTT CCC CAG ATT TCA CC-3′ (SEQ ID NO: 5); forward primer “Pr−1”: 5′-ATC TGG GGA ACT CTT CTC AG-3′(SEQ ID NO: 6); reverse primer “Pr+2(4A2)”: 5′-TAC AGT TGT TGC AGA TAC G-3′(SEQ ID NO: 7); forward primer “Pr−2(4A)”: 5′-ACA ACG TAT CTG CAA CAA CTG-3′ (SEQ ID NO: 8); reverse primer “Pr+3(4A)”: 5′-TCA GGC TTA ACT GCA GAA CC-3′ (SEQ ID NO: 9); forward primer “Pr−3(4A)”: 5′-TTG GTT CTG CAG TTA AGC C-3′ (SEQ ID NO: 10); reverse primer “Pr+2(4A1)”: 5′-AGC AGC ACA AGG GCA ATC C-3′ (SEQ ID NO: 1); reverse primer “Pr+1(4A)”: 5′-ACA GGG CTA TAT TGT GGG-3′ (SEQ ID NO: 12); forward primer “KS1X”: 5′-CCT GAG ATG CCA GCT GTC C-3′ (SEQ ID NO: 13); reverse primer “SK1XX”: 5′-CTG AAG CAT TAG AAG CCA AC-3′ (SEQ ID NO: 14); forward primer “KS2a1”: 5′-ACC TTG GAC CAG GCT GCC AG-3′ (SEQ ID NO: 15); reverse primer “SK3”5′-AGG TTT GTT CGA GTT CCA G-3′ (SEQ ID NO: 16); forward primer “KS4”: 5′-ACA ATT ACT GGC AAG TAT GG-3′ (SEQ ID NO: 17); reverse primer “SK6A”: 5′-CCT TCT CAG CCT TGC TAC C-3′ (SEQ ID NO: 18); forward primer “KS5”: 5′-ACA CCT CAG CAG ATG CTA CC-3′ (SEQ ID NO: 19); reverse primer “SK8”: 5′-ATG GAT GAC TGA CAT GGC C-3′ (SEQ ID NO: 20); forward primer “KS6”: 5′-AAG GAT GAA CAT GGT CAC C-3′ (SEQ ID NO: 21).

Families with a history of POAG in Klamath Falls, Oreg., are screened by SSCP according to the method of Hue et al., The Journal of Investigative Ophthalmology 105.4: 529-632 (1995), herein incorporated by reference). SSCP primers SK-1a, ca2, CA2, Pr+1, Pr−2(4A), Pr+3(4A), SK1XX, and KS6 detect single strand conformational polymorphisms in this population. An SSCP is detected using SSCP primers Pr+3(4A) and Pr−2(4A). 70 family members of the Klamath Fall, Oreg. are screened with these primers and the results are set forth in Table 1.

TABLE 1

| | Total | SSCP+ | SSCP− |
|---|---|---|---|
| Glaucoma positive individuals¹ | 12 | 12 | 0 |
| Glaucoma negative individuals | 13 | 0 | 13 |
| Spouses (glaucoma negative) | 16 | 2 | 14 |
| Others² | 29 | 6 | 23 |

¹glaucoma positive individuals as determined by IOP of greater than 25 mmHg
²unidentified glaucoma due to the age of the individual.

A second SSCP is detected using SSCP primers Pr+1 and CA2. 14 family members of the Klamath Fall, Oreg. are screened with these primers. A characteristic polymorphism is found in the 6 affected family members but absent in the 8 unaffected members. A third SSCP is detected using SSCP primers ca2 and sk-1a. The same 14 family members of the Klamath Fall, Oreg. that are screened with Pr+1 and CA2 are screened with ca2 and sk-1a primers. A characteristic polymorphism is found in the 6 affected family members but absent in the 8 unaffected members. A fourth SSCP is detected using SSCP primers KS6 and SKLXX. 22 family members of the Klamath Fall, Oreg. and 10 members of a Portland, Oreg. pedigree are screened with these primers. A polymorphism is found in exon 3. The results are as set forth in Table 2.

TABLE 2

| | Total | SSCP+ | SSCP− |
|---|---|---|---|
| Klamath Fall, Oregon | | | |
| Glaucoma positive individuals¹ | 3 | 3 | 0 |
| Glaucoma negative individuals | 6 | 0 | 6 |
| Others² | 13 | 6 | 7 |
| Portland, Oregon | | | |
| Glaucoma positive individuals¹ | 6 | 6 | 0 |
| Glaucoma negative individuals | 4 | 0 | 4 |
| Others² | 0 | 0 | 0 |

¹glaucoma positive individuals as determined by IOP of greater than 25 mmHg
²unidentified glaucoma due to the age of the individual.

### EXAMPLE II

### Preparation of Preferred Topical Formulations

A hydrated polymeric dispersion is prepared by slowly dispersing 1.0 part of Noveon. TM. AA-1 type acrylic polymer, available from B. F Goodrich, into a beaker fitted with an overhead stirrer containing two-thirds of the final deionized water content and stirring for one hour. Then, 0.10 parts of edetate disodium is added to the dispersion followed by stirring for 10 minutes. The resulting dispersion possessing a pH of about 3.0-3.5 is sterilized by autoclaving at 121° C. for 20 minutes. Diclofenac sodium frequently used in the treatment of ocular inflammation is dissolved separately in approximately one-fifth of the final weight of water, added to the polymer mixture by sterile filtration (0.22 μm filter) and stirred for 10 minutes. The mixture is adjusted to pH 7.2 with ION sodium hydroxide, brought to final weight with water by sterile filtration and aseptically filled into unit-dose containers.

Table 3 sets forth the amounts of each component in the sample formulations.

TABLE 3
Amount of Each Component
In Sample Formulations

| | Sample 1 | Sample 2 | Sample 3 |
|---|---|---|---|
| | Weight | Weight | Weight |
| | Percent | Percent | Percent |
| INGREDIENTS | (% w/w) | (% w/w) | (% w/w) |
| Diclofenac Sodium | 0.01 | 0.05 | 0.1 |
| Noveon ™ -AA-1 | 1.0 | 1.0 | 1.0 |
| Edetate Disodium | 0.1 | 0.1 | 0.1 |
| Sodium Chloride | 0.7 | 0.7 | 0.7 |
| Sodium Hydroxide | g.s. to | g.s. to | g.s. to |
| | pH 7.2 | pH 7.2 | pH 7.2 |
| Purified Water | 100.0 | 100.0 | 100.0 |

### EXAMPLE III

### Treatment with Diclofenac in the Presence of the TIGR mt-1 Mutation

After treatment for 6 months with varying amounts of diclofenac in the presence of the mt-1 mutation, the following results are observed:

Percentage of subjects exhibiting a Mean Diurnel IOP < 22 mm Hg
After 6 Months of Treatment

| Level of Diclofenac | |
|---|---|
| administered | % of subjects |
| 0.03% | 55% |
| 0.06% | 60% |
| 0.1% | 64% |
| vehicle (placebo) | 22% |

Percentage of Patients Exhibiting a Decrease
in Diurnal IOP >= 2 mm Hg
After 6 Months of Treatment

| Level of Diclofenac | |
|---|---|
| administered | % of subjects |
| 0.03% | 36% |
| 0.06% | 40% |
| 0.1% | 64% |
| vehicle (placebo) | 22% |

Mean Change in Diurnal IOP at 6 Months

| | mt-1 mutation | mt-1 mutation |
|---|---|---|
| | present | absent |
| Treatment with | −2.1 mm Hg | −1.5 mm Hg |
| 0.1% Diclofenac | | |
| Vehicle only | −0.3 mm Hg | −1.6 mm Hg |

### EXAMPLE IV

### Treatment with Diclofenac in the Presence of the TIGR mt-11 Mutation

The same procedures described in Example II were followed with respect to administering Diclofenac to glaucomic patients having the TIGRmt-11 mutation. The following results are observed.

Mean Change in Diurnal IOP at 6 Months

| | mt-11 mutation | mt-11 mutation |
|---|---|---|
| | present | absent |
| Treatment with | −2.0 mm Hg | −1.3 mm Hg |
| 0.1% Diclofenac | | |
| Vehicle only | −1.1 mm Hg | −1.7 mm Hg |

### EXAMPLE V

### Treatment with Diclofenac in the Presence of Both the TIGR mt-1 and mt-11 Mutations

The procedures described in examples II and III were applied to glaucomic patients possessing both the mt-1 and mt-11 mutations in the TIGR gene. The results suggest the effects of multiple polymorphisms may be cumulative.

Mean Change in Diurnal IOP at 6 Months

| | mt-1 and mt-11 | mt-1 and mt-11 |
|---|---|---|
| | mutations present | mutations absent |
| Treatment with | −2.9 mm Hg | −0.6 mm Hg |
| 0.1% Diclofenac | | |
| Vehicle only | +0.4 mm Hg | −1.8 mm Hg |

### EXAMPLE VI

### The Use of TIGRmt1 to Identify Progressive IOP Disease

Clinical trials are conducted to show the ability of TIGRmt1 to identify individuals with ocular hypertension who exhibit a real and progressive increase in IOP. It is shown that, instead of the expected 1 to 2 mm Hg “white coat effect” in which the placebo group IOP drops, those with TIGRmt1 positive status exhibit an average increase in IOP of 1.4 mm Hg (see FIG. 2). In contrast, those who were identified as not having the TIGRmt1 mutation exhibited a decrease of IOP over a six month period that was greater than that exhibited by the entire group.

Ocular hypertension is determined by measuring the intraocular pressure of the subject using a Goldman tonometer or direct pressure device. Two tissue samples are collected by buccal swab (right inner cheek and left inner cheek) from each ocular hypertensive subject. Samples are sent to a genetic testing laboratory for analysis. Prevalence of the TIGRmt-1 and mt-11 mutations in the population of ocular hypertensives is determined from the number of subjects with these mutations compared to the frequencies with those reported in the literature for the normal population and demographics are evaluated by summarizing demographic and family history data for subjects with and without the TIGR mutations.

The above discussion of this invention is directed primarily to preferred embodiments and practices thereof. It will be readily apparent to those skilled in the art that further changes and modifications in actual implementation of the concepts described herein can easily be made without departing from the spirit and scope of the invention as defined by the following claims. Each reference cited herein is hereby incorporated by reference in its entirety.

We claim:

1. A method of providing treatment to a glaucomatous patient comprising:
(a) determining whether said patient has a mutation in the TIGR promoter selected from the group consisting of TIGRmt1, TIGRmt11, or both TIGRmt1 and TIGRmt11; and
(b) administering an effective amount of a non-steroidal anti-inflammatory drug to said patient.

2. The method of claim 1, wherein said glaucoma is an inherited glaucoma.

3. The method of claim 2, wherein said glaucoma is an open angle glaucoma.

4. The method of claim 3, wherein said open angle glaucoma is primary open angle glaucoma.

5. The method of claim 1, where said mutation is determined through the use of nucleic acid amplification methods.

6. The method of claim 1, where said mutation is determined through the use of a marker nucleic acid molecule.

7. The method of claim 1, wherein said mutation is determined using a restriction endonuclease.

8. The method of claim 1, where said mutation is determined using oligonucleotide probes.

9. The method of claim 1, wherein said non-steroidal anti-inflamatory drug is administered using a polymeric carrier.

10. The method of claim 9, wherein said polymeric carrier is a lightly crosslinked polymer of acrylic acid.

11. The method of claim 10, wherein said lightly crosslinked polymer is prepared in an aqueous suspension.

12. The method of claim 1, wherein said non-steroidal anti-inflammatory drug is administered in a topical formulation.

13. The method of claim 1, wherein said non-steroidal anti-inflammatory drug is administered in an ocular injection.

14. The method of claim 1, wherein said non-steroidal anti-inflammatory drug is administered orally.

15. The method of claim 1, wherein said non-steroidal anti-inflammatory drug is a cyclooxygenase inhibiting agent.

16. The method of claim 15, wherein said cyclooxygenase inhibiting agent is selected from the group consisting of salicylates, fenamates, indoles, phenylalkanoic acids, phenylacetic acids, and pyrazolons.

17. The method of claim 15, wherein said cyclooxygenase inhibiting agent is selected from the group consisting of aspirin, salicylic acid, diflunisol, indomethacin, sulinda, tolmetin, fenoprofen, flurbiprofen, ibuprofen, ketoprofen, ketorolac, naproxen, piroxicam, suprofen, diclofenac, oxyphenbutazone, phenylbutazone, antipyrine, aminopyrine, and azapropazone.

18. The method of claim 16, wherein said cyclooxygenase inhibiting agent is a phenylacetic acid.

19. The method of claim 18, wherein said phenylacetic acid is diclofenac.

20. The method of claim 1, wherein said effective amount is greater than about 0.01% by weight of a non-steroidal anti-inflammatory drug.

21. The method of claim 20, wherein said effective amount is greater than about 0.03% by weight of a non-steroidal anti-inflammatory drug.

22. The method of claim 21, wherein said effective amount is greater than about 0.06% by weight of a non-steroidal anti-inflammatory drug.

23. The method of claim 22, wherein said effective amount is greater than about 0.1% by weight of a non-steroidal anti-inflammatory drug.

24. A method of preventing increases in intraocular pressure in a patient predisposed to developing glaucoma comprising:
(a) determining whether said patient has a mutation in the TIGR gene selected from the group consisting of TIGRmt1, TIGRmt11, or both TIGRmt1 and TIGRmt11; and
(b) administering an effective amount of a non-steroidal anti-inflammatory drug to said patient.

25. The method of claim 24, wherein said patient is asymptomatic of glaucoma.

26. The method of claim 24, wherein said intraocular pressure is below about 25 mm Hg.

27. The method of claim 26, wherein said intraocular pressure is below about 22 mm Hg.

28. The method of claim 27, wherein said intraocular pressure is below about 20 mm Hg.

29. The method of claim 24, wherein said glaucoma is an inherited glaucoma.

30. The method of claim 29, wherein said glaucoma is an open angle glaucoma.

31. The method of claim 30, wherein said open angle glaucoma is primary open angle glaucoma.

32. The method of claim 24, where said mutation is determined through the use of nucleic acid amplification methods.

33. The method of claim 24, where said mutation is determined through the use of a marker nucleic acid molecule.

34. The method of claim 24, wherein said mutation is determined using a restriction endonuclease.

35. The method of claim 24, where said mutation is determined using oligonucleotide probes.

36. The method of claim 24, wherein said non-steroidal anti-inflamatory drug is administered using a polymeric carrier.

37. The method of claim 36, wherein said polymeric carrier is a lightly crosslinked polymer of acrylic acid.

38. The method of claim 37, wherein said lightly crosslinked polymer is prepared in an aqueous suspension.

39. The method of claim 24, wherein said non-steroidal anti-inflammatory drug is administered in a topical formulation.

40. The method of claim 24, wherein said non-steroidal anti-inflammatory drug is administered in an ocular injection.

41. The method of claim 24, wherein said non-steroidal anti-inflammatory drug is administered orally.

42. The method of claim 24, wherein said non-steroidal anti-inflammatory drug is a cyclooxygenase inhibiting agent.

43. The method of claim 42, wherein said cyclooxygenase inhibiting agent is selected from the group consisting of salicylates, fenamates, indoles, phenylalkanoic acids, phenylacetic acids, and pyrazolons.

44. The method of claim 42, wherein said cyclooxygenase inhibiting agent is selected from the group consisting of aspirin, salicylic acid, diflunisol, indomethacin, sulinda, tolmetin, fenoprofen, flurbiprofen, ibuprofen, ketoprofen, ketorolac, naproxen, piroxicam, suprofen, diclofenac, oxyphenbutazone, phenylbutazone, antipyrine, aminopyrine, and azapropazone.

45. The method of claim 43, wherein said cyclooxygenase inhibiting agent is a phenylacetic acid.

46. The method of claim 45, wherein said phenylacetic acid is diclofenac.

47. The method of claim 24, wherein said effective amount is greater than about 0.01% by weight of a non-steroidal anti-inflammatory drug.

48. The method of claim 47, wherein said effective amount is greater than about 0.03% by weight of a non-steroidal anti-inflammatory drug.

49. The method of claim 48, wherein said effective amount is greater than about 0.06% by weight of a non-steroidal anti-inflammatory drug.

50. The method of claim 49, wherein said effective amount is greater than about 0.1% by weight of a non-steroidal anti-inflammatory drug.

51. A method for decreasing the mean diurnal intra-ocular pressure in a patient, comprising
(a) identifying the presence of at least one mutation in the TIGR gene selected from the group consisting of TIGRmt1, TIGRmt11, or both TIGRmt1 and TIGRmt11; and
(b) administering an effective amount of a non-steroidal anti-inflammatory drug to said patient.

52. The method of claim 51, wherein said non-steroidal anti-inflammatory drug is cyclooxygenase inhibiting agent.

53. The method of claim 52, wherein said cyclooxygenase inhibiting agent is selected from the group consisting of salicylates, fenamates, indoles, phenylalkanoic acids, phenylacetic acids, and pyrazolons.

54. The method of claim 52, wherein said cyclooxygenase inhibiting agent is selected from the group consisting of aspirin, salicylic acid, diflunisol, indomethacin, sulinda, tolmetin, fenoprofen, flurbiprofen, ibuprofen, ketoprofen, ketorolac, naproxen, piroxicam, suprofen, diclofenac, oxyphenbutazone, phenylbutazone, antipyrine, aminopyrine, and azapropazone.

55. The method of claim 53, wherein said cyclooxygenase inhibiting agent is a phenylacetic acid.

56. The method of claim 55, wherein said phenylacetic acid is diclofenac.

57. The method of claim 51, wherein said decrease in mean diurnal intra-ocular pressure is greater than about 1 mm Hg per 6 months.

58. The method of claim 57, wherein said decrease in mean diurnal intra-ocular pressure is greater than about 2.5 mm Hg per 6 months.

59. The method of claim 58, wherein said decrease in mean diurnal intra-ocular pressure is greater than about 3 mm Hg per 6 months.

60. The method of claim 49, wherein said effective amount is greater than about 0.01% of a non-steroidal anti-inflammatory drug by weight.

61. The method of claim 60, wherein said effective amount is greater than about 0.03% of a non-steroidal anti-inflammatory drug by weight.

62. The method of claim 61, wherein said effective amount is greater than about 0.06% of a non-steroidal anti-inflammatory drug by weight.

63. The method of claim 62, wherein said effective amount is greater than about 0.1% of a non-steroidal anti-inflammatory drug by weight.

64. The method of claim 49, wherein said glaucoma is an inherited glaucoma.

65. The method of claim 64, wherein said glaucoma is an open angle glaucoma.

66. The method of claim 92, wherein said open angle glaucoma is primary open angle glaucoma.

67. The method of claim 49, where said mutation is determined through the use of nucleic acid amplification methods.

68. The method of claim 49, where said mutation is determined through the use of a marker nucleic acid molecule.

69. The method of claim 49, wherein said mutation is determined using a restriction endonuclease.

70. The method of claim 49, where said mutation is determined using oligonucleotide probes.

71. The method of claim 49, wherein said non-steroidal anti-inflamatory drug is administered using a polymeric carrier.

72. The method of claim 71, wherein said polymeric carrier is a lightly crosslinked polymer of acrylic acid.

73. The method of claim 72, wherein said lightly crosslinked polymer is prepared in an aqueous suspension.

74. The method of claim 49, wherein said non-steroidal anti-inflammatory drug is administered in a topical formulation.

75. The method of claim 49, wherein said non-steroidal anti-inflammatory drug is administered in an ocular injection.

76. The method of claim 49, wherein said non-steroidal anti-inflammatory drug is administered orally.

77. A method of providing treatment to a patient known to possess at least one mutation in the TIGR gene selected from the group consisting of TIGRmt1 and TIGRmt11 comprising:
(a) administering an effective amount of a non-steroidal anti-inflammatory drug to said patient; and
(b) monitoring the level of intraocular pressure of said patient.

78. The method of claim 77, wherein said non-steroidal anti-inflammatory drug is a cyclooxygenase inhibiting agent.

79. The method of claim 78, wherein said cyclooxygenase inhibiting agent is selected from the group consisting of salicylates, fenamates, indoles, phenylalkanoic acids, phenylacetic acids, and pyrazolons.

80. The method of claim 78, wherein said cyclooxygenase inhibiting agent is selected from the group consisting of aspirin, salicylic acid, diflunisol, indomethacin, sulinda, tolmetin, fenoprofen, flurbiprofen, ibuprofen, ketoprofen, ketorolac, naproxen, piroxicam, suprofen, diclofenac, oxyphenbutazone, phenylbutazone, antipyrine, aminopyrine, and azapropazone.

81. The method of claim 79, wherein said cyclooxygenase inhibiting agent is a phenylacetic acid.

82. The method of claim 81, wherein said phenylacetic acid is diclofenac.

83. The method of claim 77, wherein said effective amount is greater than about 0.01% of a non-steroidal anti-inflammatory drug by weight.

84. The method of claim 83, wherein said effective amount is greater than about 0.03% of a non-steroidal anti-inflammatory drug by weight.

85. The method of claim 84, wherein said effective amount is greater than about 0.06% of a non-steroidal anti-inflammatory drug by weight.

86. The method of claim 85, wherein said effective amount is greater than about 0.1% of a non-steroidal anti-inflammatory drug by weight.

87. The method of claim 77, wherein said level of intraocular pressure is below about 25 mm Hg after six months.

88. The method of claim 87, wherein said level of intraocular pressure is below about 22 mm Hg after six months.

89. The method of claim 88, wherein said level of intraocular pressure is below about 20 mm Hg after six months.

90. The method of claim 77, wherein said patient is asymptomatic for glaucoma.

91. The method of claim 77, wherein said glaucoma is an inherited glaucoma.

92. The method of claim 91, wherein said glaucoma is an open angle glaucoma.

93. The method of claim 92, wherein said open angle glaucoma is primary open angle glaucoma.

94. The method of claim 77, where said mutation is determined through the use of nucleic acid amplification methods.

95. The method of claim 77, where said mutation is determined through the use of a marker nucleic acid molecule.

96. The method of claim 77, wherein said mutation is determined using a restriction endonuclease.

97. The method of claim 77, where said mutation is determined using oligonucleotide probes.

98. The method of claim 77, wherein said non-steroidal anti-inflamatory drug is administered using a polymeric carrier.

99. The method of claim 98, wherein said polymeric carrier is a lightly crosslinked polymer of acrylic acid.

100. The method of claim 99, wherein said lightly crosslinked polymer is prepared in an aqueous suspension.

101. The method of claim 77, wherein said non-steroidal anti-inflammatory drug is administered in a topical formulation.

102. The method of claim 77, wherein said non-steroidal anti-inflammatory drug is administered in an ocular injection.

103. The method of claim 77, wherein said non-steroidal anti-inflammatory drug is administered orally.

104. A method of selecting a patient for a clinical trial involving a treatment for elevated intraocular pressure, comprising:
determining whether said patient has either the TIGRmt1 mutation, or both the TIGRmt1 and TIGRmt11 mutations; and,
selecting said patient if said patient has said TIGRmt1 mutation or both of said TIGRmt1 and TIGRmt11 mutations.

105. The method of claim 104, wherein said treatment comprises administration of a medicament to said patient.

106. The method of claim 104, further comprising the step of not selecting said patient if said patient lacks said TIGRmt1 mutation.

107. The method of claim 104, wherein said mutation is said TIGRmt1.

108. A method of improving the results of a drug study involving a treatment for elevated intraocular pressure, comprising:
a) determining whether a candidate patient for inclusion in said study has either the TIGRmt1 mutation, or both the TIGRmt1 and TIGRmt11 mutations; and,
b) selecting said patient for inclusion in said study if said patient has said TIGRmt1 mutation or both of said TIGRmt1 and TIGRmt11 mutations.

109. The method of claim 108, further comprising repeating steps a) and b) until a sufficient number of patients for said study have been selected.

110. The method of claim 108, further comprising the step of not selecting said patient if said patient lacks said TIGRmt1 mutation.

111. The method of claim 108, wherein said mutation is said TIGRmt1.

112. A method of determining whether a patient is at risk for developing elevated intraocular pressure, comprising:
determining whether said patient has either the TIGRmt1 mutation, or both the TIGRmt1 and TIGRmt11 mutations; and,
determining that said patient is at risk for developing elevated intraocular pressure if said patient has said TIGRmt1 mutation or both of said TIGRmt1 and TIGRmt11 mutations.

113. The method of claim 112, further comprising administering a medicament to said patient to prevent an increase in intraocular pressure.

114. The method of claim 104, wherein said mutation is said TIGRmt1.

115. A method of testing the efficacy of a therapeutic agent at counteracting glaucoma pathogenic mechanisms, comprising:
a) determining whether a candidate patient for inclusion in a study has either the TIGRmt1 mutation, or both the TIGRmt1 and TIGRmt11 mutations;
b) selecting said patient for inclusion in said study if said patient has said TIGRmt1 mutation or both of said TIGRmt1 and TIGRmt11 mutations;
c) repeating steps a) and b) one or more times; and,
d) testing said agent in said study.

116. The method of claim 115, wherein said agent comprises a member selected from the group consisting of prostaglandin inhibitors, selective or non-selective COX 1 or COX 2 inhibitors, antioxidants, neuroprotective agents, platelet activating factor antagonists, alpha agonists, beta blockers, beta agonists, prostaglandin agonists, hypotensive lipids, carbonic anhydrase inhibitors, cholinergic agents, and combinations of the foregoing.
